(19) United States
(12) Patent Application Publication
(10) Pub. No.: US 2011/0177606 A1
(43) Pub. Date: Jul. 21, 2011

(54) DETECTION OF TRINITROTOLUENE

(75) Inventors: Itamar Willner, Mevasseret Zion (IL); Ran Tel-Vered, Jerusalem (IL); Michael Riskin, Jerusalem (IL)

(73) Assignee: Yissum Research Development Company of the Hebrew University of Jerusalem, Ltd., Jerusalem (IL)

(21) Appl. No.: 13/001,972

(22) PCT Filed: Jun. 30, 2009
(86) PCT No.: PCT/IL2009/000656

§ 371 Date: Apr. 7, 2011

Related U.S. Application Data
(60) Provisional application No. 61/076,970, filed on Jun. 30, 2008.

(30) Foreign Application Priority Data
Jun. 15, 2009 (IL) 199354

(51) Int. Cl. G01N 33/22; G01N 27/00; B82Y 30/00

(52) U.S. Cl. 436/110; 422/82.02; 977/773

(57) ABSTRACT

An ultrasensitive method for detecting analytes in a sample is provided. The method involves the use of a matrix of nanoparticles which are associated with recognition groups capable of undergoing interaction with the analyte.

### FIELD OF THE INVENTION

This invention relates to a method and device for detecting trinitrotoluene.

### BACKGROUND OF THE INVENTION

Sensors for the detection of explosives are important for various disciplines including humanitarian de-mining, remediation of explosives waste sites, homeland security, and forensic applications. Different sensors for analyzing explosives and, specifically, nitrobenzene (or nitrotoluene) derivatives were reported in the past decade. These included optical sensors where the fluorescence of functional polymers was quenched by the nitroaromatic compounds [1,2] luminescent polymer nanoparticles, such as polysilole, that were quenched by trinitrotoluene (TNT) [3] or fluorescent silicon nanoparticles that were quenched by nitroaromatic vapors [4].

The redox activity of the nitro groups associated with many of the explosives was used to develop electrochemical sensors [5], and modified electrodes such as mesoporous SiO₂-functionalized electrodes were employed to enhance the sensitivity of detection of nitroaromatic explosives [6]. Other electronic devices for the analysis of explosives included surface acoustic wave (SAW) systems. The coating of the piezoelectric devices with silicon polymers carbowax or cyclodextrin polymers yielded functional coatings that enabled the electronic transduction of explosives adsorbed to these matrices. The eliciting of antibodies that exhibit specific binding to nitroaromatics enabled the development of biosensors for explosives, using immunocomplexes as sensing units. This was exemplified with the development of TNT biosensors based on the displacement of the anti-TNT antibody from a surface-confined immunocomplex by TNT and the transduction of the dissociation of the antibody by surface plasmon resonance (SPR) spectroscopy [7,8] or quartz crystal microbalance (QCM) measurements [7]. Also, a quantum dot-based fluorescent biosensor was developed by the application of antibody-functionalized quantum dots as reporter units. The association of a quencher-TNT conjugate to the antibody resulted in the FRET quenching of the quantum dots, and the displacement of the conjugate by TNT regenerated the fluorescence of the quantum dots [9]. Although substantial progress was achieved in the sensing of explosives, the different analytical protocols suffer from insufficient sensitivity, lack of specificity, long analysis time intervals, and/or complex and expensive analytical protocols.

The unique electronic and optical properties of metallic and semiconductor nanoparticles, NPs, added new dimensions to the area of sensors. The aggregation of Au NPs as a result of sensing events and the formation of an interparticle coupled plasmon absorbance was used for the development of colorimetric sensors [10]. For example, color changes as a result of aggregation of Au nanoparticles were used to detect phosphatase activity [11], polynucleotides [12], or alkali (lithium) [13] ions. Also, the shifts in the plasmonic absorption bands associated with Au nanoclusters as a result of changes in the surface dielectric properties upon sensing were used to develop optical sensors for dopamine [14], adrenaline [15], cholesterol [16], DNA hybridization [17], and pH changes [18]. The layer-by-layer deposition of Au NPs on electrodes by the electrostatic cross-linking of the NPs by charged molecular receptors was used to construct electrochemical sensors for different neurotransmitters [19].

The imprinting of molecular recognition in organic or inorganic polymer matrices is known to permit generation of selective binding sites for the imprinted substrates [20]. Indeed, numerous optical [21] or electronic [22] sensors based on imprinted polymer matrices have been developed in the past two decades. For example, electrochemical sensors that consisted of imprinted organic [23] or inorganic [24] polymers were developed, and imprinted inorganic matrices associated with the gate surface of field-effect transistors were applied for the stereoselective or chiroselective analysis of the imprinted substrates [25]. Similarly, a quartz crystal microbalance [26] and surface plasmon resonance spectroscopy [27] were used as readout methods for the binding of substrates to the imprinted sites. The use of imprinted polymers as functional sensing matrices suffers, however, from several basic limitations. The density of imprinted sites is relatively low, and thus, for sensitive sensing thick polymer matrices are required. This leads, however, to slow binding of the analytes to the recognition sites (long analysis time intervals) and to inefficient communication between the binding sites and the transducers. In fact, several studies suggested the use of imprinted monolayers [26], multilayers [27], and thin films to overcome these difficulties.

### REFERENCES

[1] (a) Swager, T. M. Acc. Chem. Res. 1998, 31, 201-207. (b) McQuade, D. T.; Pullen, A. E.; Swager, T. M. Chem. ReV. 2000, 100, 2537-2574. (c) Yang, J.-S.; Swager, T. M. J. Am. Chem. Soc. 1998, 120, 11864-11873.
[2] (a) Chang, C.-P.; Chao, C.-Y.; Huang, J.-H.; Li, A.-K.; Hsu, C.-S.; Lin, M.-S.; Hsieh, B.-R.; Su, A.-C. Synth. Met. 2004, 144, 297-301. (b) Sohn, H.; Sailor, M. J.; Magde, D.; Trogler, W. C. J. Am. Chem. Soc. 2003, 125, 3821-3830.
[3] (a) Chen, J.; Law, C. C. W.; Lam, J. W. Y.; Dong, Y.; Lo, S. M. F.; Williams, I. D.; Zhu, D.; Tang, B. Z. Chem. Mater. 2003, 15, 1535-1546. (b) Toal, S. J.; Magde, D.; Trogler, W. C. Chem. Commun. 2005, 5465-5467.
[4] Content, S.; Trogler, W. C.; Sailor, M. J. Chem. sEur. J. 2000, 6, 2205-2213.
[5] (a) Wang, J.; Bhada, R. K.; Lu, J.; MacDonald, D. Anal. Chim. Acta 1998, 361, 85-91. (b) Wang, J.; Hocevar, S. B.; Ogorevc, B. Electrochem. Commun. 2004, 6, 176-179. (c) Hrapovic, S.; Majid, E.; Liu, Y.; Male, K.; Luong, J. H. T. Anal. Chem. 2006, 78, 5504-5512. (d) Zhang, H.-X.; Hu, J.-S.; Yan, C.-J.; Jiang, L.; Wan, L.-J. Phys. Chem. Chem. Phys. 2006, 8, 3567-3572.
[6] Zhang, H.-X.; Cao, A.-M.; Hu, J.-S.; Wan, L.-J.; Lee, S.-T. Anal. Chem. 2006, 78, 1967-1971.
[7] Larsson, A.; Angbrant, J.; Ekeroth, J.; Mansson, P.; Liedberg, B. Sens. Actuators, B 2006, 113, 730-748.
[8] Shankaran, D. R.; Gobi, K. V.; Sakai, T.; Matsumoto, K.; Toko, K.; Miura, N. Biosens. Bioelectron. 2005, 20, 1750-1756.
[9] Goldman, E. R.; Medintz, I. L.; Whitley, I. L.; Hayhurst, A.; Clapp, A. R.; Uyeda, H. T.; Deschamps, J. R.; Lassman, M. E.; Mattoussi, H. J. Am. Chem. Soc. 2005, 127, 6744-6751.
[10] (a) Daniel, M.-C.; Astruc, D. Chem. ReV. 2004, 104, 293-346. (b) Rosi, N. L.; Mirkin, C. A. Chem. Rev. 2005, 105, 1547-1562.
[11] Choi, Y.; Ho, N.-H.; Tung, C.-H. Angew. Chem., Int. Ed. 2007, 46, 707-709.
[12] Storhoff, J. J.; Elghanian, R.; Mucic, R. C.; Mirkin, C. A.; Letsinger, R. L. J. Am. Chem. Soc. 1998, 120, 1959-1964.
[13] Obare, S. O.; Hollowell, R. E.; Murphy, C. J. Langmuir 2002, 18, 10407-10410.
[14] Matsui, J.; Akamatsu, K.; Hara, N.; Miyoshi, D.; Nawafune, H.; Tamaki, K.; Sugimoto, N. Anal. Chem. 2005, 77, 4282-4285.
[15] Matsui, J.; Akamatsu, K.; Nishiguchi, S.; Miyoshi, D.; Nawafune, H.; Tamaki, K.; Sugimoto, N. Anal. Chem. 2004, 76, 1310-1315.
[16] Tokareva, I.; Tokarev, I.; Minko, S.; Huffer, E.; Fendler, J. H. Chem. Commun. 2006, 31, 3343-3345.
[17] Hu, W. P.; Chen, S.-J.; Huang, K.-T.; Hsu, J. H.; Chen, W. Y.; Chang, G. L.; Lai, K.-A. Biosens. Bioelectron. 2004, 19, 1465-1471.
[18] Jiang, G.; Baba, A.; Ikarashi, H.; Xu, R.; Locklin, J.; Kashif, K. R.; Shinbo, K.; Kato, K.; Kaneko, F.; Advincula, R. J. Phys. Chem. C 2007, 111, 18687-18694.
[19] (a) Shipway, A. N.; Lahav, M.; Willner, I. AdV. Mater. 2000, 12, 993-998. (b) Lahav, M.; Gabai, R.; Shipway, A. N.; Willner, I. Chem. Commun. 1999, 19, 1937-1938. (c) Shipway, A. N.; Lahav, M.; Blonder, R.; Willner, I. Chem. Mater. 1999, 11, 13-15. (d) Lahav, M.; Shipway, A. N.; Willner, I.; Nielsen, M. B.; Stoddart, J. F. J. Electroanal. Chem. 2000, 482, 217-221.
[20] (a) Wulff, G. Angew. Chem., Int. Ed. 1995, 34, 1812-1832. (b) Wulff, G. Chem. ReV. 2002, 102, 1-27. (c) Mosbach, K. Trends Biochem. Sci. 1994, 19, 9-14. (d) Haupt, K.; Mosbach, K. Chem. ReV. 2000, 100, 2495-2504. (e) Bossi, A.; Bonini, F.; Turner, A. P. F.; Piletsky, S. A. Biosens. Bioelectron. 2007, 22, 1131-1137.
[21] (a) Suarez-Rodriguez, J. L.; Diaz-Garcia, M. E. Biosens. Bioelectron. 2001, 16, 955-961. (b) Surugiu, I.; Svitel, J.; Ye, L.; Haupt, K.; Danielsson, B. Anal. Chem. 2001, 73, 4388-4392. (c) Wang, W.; Gao, S.; Wang, B. Org. Lett. 1999, 1, 1209-1212. (d) Hu, X.; An, Q.; Li, G.; Tao, S.; Liu, J. Angew. Chem., Int. Ed. 2006, 45, 8145-8148.
[22] (a) Liang, H.-J.; Ling, T.-R.; Rick, J. F.; Chou, T.-C. Anal. Chim. Acta 2005, 542, 83-89. (b) Kirsch, N.; Hart, J. P.; Bird, D. J.; Luxton, R. W.; McCalley, D. V. Analyst 2001, 126, 1936-1941.
[23] (a) Weng, C.-H.; Yeh, W.-M.; Ho, K.-C.; Lee, G.-B. Sens. Actuators, B 2007, B121, 576-582. (b) Shoji, R.; Takeuchi, T.; Kubo, I. Anal. Chem. 2003, 75, 4882-4886.
[24] (a) Zhou, Y.; Yu, B.; Shiu, E.; Levon, K. Anal. Chem. 2004, 76, 2689-2693. (b) Fireman-Shoresh, S.; Turyan, I.; Mandler, D.; Avnir, D.; Marx, S. Langmuir 2005, 21, 7842-7847. (c) Lahav, M.; Kharitonov, A. B.; Katz, O.; Kunitake, T.; Willner, I. Anal. Chem. 2001, 73, 720-723.
[25] (a) Zayats, M.; Lahav, M.; Kharitonov, A. B.; Willner, I. Tetrahedron 2002, 58, 815-824. (b) Pogorelova, S. P.; Zayats, M.; Bourenko, T.; Kharitonov, A. B.; Lioubashevski, O.; Katz, E.; Willner, I. Anal. Chem. 2003, 75, 509-517.
[26] (a) Pogorelova, S. P.; Bourenko, T.; Kharitonov, A. B.; Willner, I. Analyst 2002, 127, 1484-1491. (b) Cao, L.; Li, S. F. Y.; Zhou, X. C. Analyst 2001, 126, 184-188.
[27] (a) Matsunaga, T.; Hishiya, T.; Takeuchi, T. Anal. Chim. Acta 2007, 591, 63-67. (b) Li, X.; Husson, S. M. Biosens. Bioelectron. 2006, 22, 336-348. (c) Raitman, O. A.; Chegel, V. I.; Kharitonov, A. B.; Zayats, M.; Katz, E.; Willner, I. Anal. Chim. Acta 2004, 504, 101-111.
[28] Gao, D.; Zhang, Z.; Wu, M.; Xie, C.; Guan, G.; Wang, D. J. Am. Chem. Soc. 2007, 129, 7859-7866.
[29] Hu, X.; An, Q.; Li, G.; Tao, S.; Liu, J. Angew. Chem., Int. Ed. 2006, 45, 8145-8148.

### SUMMARY OF THE INVENTION

The present invention, in most general terms, provides use of nanoparticle matrices for ultra sensitive and selective detection of trinitrotoluene (TNT) as an exemplary nitro compound, particularly nitro aromatic compounds.

In one aspect of the invention, there is provided a method for determining the presence and/or concentration of analyte molecules in a sample, said method comprising contacting a matrix of a plurality of transition metal nanoparticles (TMNPs), each carrying a plurality of recognition groups, with a sample suspected of containing analyte molecules (TNT or any other nitro compound or a combination thereof), and monitoring at least one of a chemical and a physical change in said matrix, i.e., resulting from an interaction between said analyte molecules and said matrix, via the recognition groups, wherein said at least one of a chemical and a physical change is indicative of at least one of presence and quantity of said analyte (TNT or other nitro compound or combination thereof) in the sample.

The matrix is composed of TMNPs associated with each other through a plurality of recognition groups being carried on their surface. As each nanoparticle may form more than one bond with a neighboring nanoparticle, as further disclosed below, a net is formed having a multitude of analyte-recognition fields (in the form of cavities) that are complementary in shape and/or size to the analyte molecules to be detected. Typically, the matrix is a three-dimensional structure.

The analyte-recognition fields constitute cavities within the matrix, suitable for holding/binding the analyte molecules therein, thereby permitting at least one interaction between the analyte molecules and the recognition groups. The analyte-recognition fields may be of any size and shape and, in some embodiments, may be tailored to suit a particular molecular shape and size, as further disclosed hereinbelow. The plurality of TMNPs in the matrix are associated with each other through a plurality of recognition groups, each group linking at least two TMNPs, thereby forming the boundaries of the analyte-recognition fields in the matrix.

The groups linking the TMNPs are referred to as “recognition groups” for having the ability to chemically and/or physically interact with the analyte molecules (TNT or other nitro compounds), thereby ensuing their recognition. The recognition groups are so selected to permit recognition of a single molecular shape and/or size, a family of compounds having a distinct shape or chemical constitution (e.g., having aromatic groups, or nitro groups or a combination thereof), or a class of compounds identified by their ability to undergo chemical interaction (i.e., chemical reaction) when in the matrix. Thus, the purpose of the recognition groups is not only to provide a net having a plurality of analyte-recognition fields around the TMNPs, but also permit interaction (reversible or permanent) with the analyte molecules which enter the analyte-recognition fields, as further disclosed below.

The recognition groups are selected to undergo chemical and/or physical interaction with the analyte molecules (one or more) present in the analyte-recognition fields. Such an interaction may be through a single, double or triple bond, or through one or more of van der Waals, hydrogen bonding, π-stacking, electrostatic interaction, complexation, caging and other weak physical interactions as known in the art. In some embodiments, the physical interaction is reversible.

In some embodiments, where the recognition groups are selected to undergo physical interaction with analyte molecules having π-electron rich groups, e.g., aromatic groups, the recognition groups are selected to include one or more aromatic or electron rich moiety to permit interaction with said analyte molecules via π-stacking or other π-π interaction.

In some embodiments, the recognition groups are selected to have certain length and substitution so as to predefine the shape and size of the analyte-recognition fields formed between the TMNPs. Typically, the longer the recognition groups are, the bigger the fields which are formed; the more substituted the recognition groups are, the denser or more crowded the fields are.

The recognition groups are typically selected to maintain strong and, in some embodiments, permanent (irreversible) interaction (association, bonding) with the TMNPs. Such association is dependent on the nature of the TMNPs, their size and to a lesser extent, in some embodiments, also on the method employed for achieving association between the TMNPs and the recognition groups. In some embodiments, the recognition groups are residues of “electropolymerizable groups”, namely groups which association (e.g., covalent bonding) with the TMNPs is achieved, at least partially, through electropolymerization.

The TMNPs are nanoparticles of at least one transition metal selected from the d-block of the Periodic Table of the Elements. In some embodiments, nanoparticles are of a metal selected from platinum (Pt), palladium (Pd), iridium (Ir), gold (Au), silver (Ag), nickel (Ni) and titanium (Ti), or alloys thereof. In some embodiments, the TMNPs are gold nanoparticles. In some embodiments, the TMNPs contain gold metal and at least one additional transition metal, at least one non-metal or at least one metal (not a transition metal).

The TMNPs forming the matrix may be a mixture of two or more nanoparticle types, each may be of a different metal or metal alloy, different size, different shape, etc. In some embodiments, the matrix is composed of a mixture of gold nanoparticles and other metallic particles. In other embodiments, the matrix is composed of nanoparticles of various metals. In still other embodiments, the matrix is composed solely of gold nanoparticles.

The TMNPs may be of any shape, such as spherical, elongated, cylindrical, or in the form of amorphous nanoparticles. The TMNPs typically have at least one dimension (diameter, width) in the range of about 1 nm to 1000 nm. In some embodiments, each TMNP is, on average, of a nanometer scale (size), ranging between 1 nanometer to 1000 nanometer; between 1 nanometer and 500 nanometers; between 1 nanometer and 250 nanometers; between 1 nanometer and 250 nanometers; between 1 nanometer and 150 nanometers; between 1 nanometer and 100 nanometers; between 1 nanometer and 50 nanometers; between 1 nanometer and 25 nanometers; between 1 nanometer and 10 nanometers and between 1 nanometer and 5 nanometers. In some further embodiments, each TMNP is, on average, 1, 2, 3, 4, 5, 6, 7, 8, 9, 10 nanometers in diameter, or any intermediate diameter, e.g., 1.1, 1.2, 1.3 . . . 2.1, 2.2, 2.3 . . . 3.1, 3.2, 3.3 . . . etc.

As stated above, the matrix comprises a plurality of TMNPs, each being associated with one another through one or more recognition moieties. Such recognition moieties may have one or more reactive groups which are capable of undergoing interaction with the nanoparticles. Non-limiting examples of such reactive groups are —S, —NH₂ and —CO₂⁻. In some embodiments, where the matrix comprises or is entirely composed of gold nanoparticles, the recognition groups may be selected to have one or more reactive groups which are capable of undergoing interaction with the gold nanoparticles. In such embodiments, the one or more reactive groups are sulfur containing groups, particularly thiols. In some embodiments, the thiols are selected to amongst aromatic thiols or alkyl thiols having at least one aromatic substituent. Non-limiting examples of such sulfur containing recognition groups are thioaniline, thioaniline dimer and oligomers thereof.

In some embodiments, the recognition groups having one or more sulfur-containing groups are selected from p-thioaniline and the oligo-thionilines having 2, 3, 4, 5, 6, 7, 8, 9 or 10 p-thioaniline monomer units. In further embodiments, the recognition groups are electorpolymerized thioanilines. In some embodiments, the recognition groups is the thioaniline dimer 4-amino-3-(4-mercaptophenylamino) benzenthiol, i.e., wherein the terminal —S-aryl groups undergo association with the gold nanoparticles.

It should be noted that each TMNP may further be functionalized to affect a change (increase, decrease or substantially maintain an intrinsic property of the nanoparticle) in one or more property associated with the nanoparticles, such property may be physical or chemical and may be selected from solubility, film forming properties, aggregation, reactivity, stickiness, stabilization, reusability, adhesion, charge, interaction with a medium, and other known properties. In some embodiments, the TMNPs are functionalized to increase their solubility in a liquid medium, e.g., an aqueous medium. In other embodiments, the TMNPs are functionalized to increase their shelf-life and reusability in the matrix of the invention. In some further embodiments, the TMNPs are functionalized with negatively or positively charged functional groups. In additional embodiments, the TMNPs are functionalized with sulfonic acid containing groups. A non-limiting example of a sulfonic acid group is 2-mercaptoethane sulfonic acid or an anion thereof.

In some additional embodiments, the TMNPs are functionalized with monomers of the recognition groups which have not undergone polymerization and subsequent association with neighboring TMNPs.

In some embodiments, the TMNP matrix is bound to an active surface which, in some embodiments, is conductive and thus capable of reporting at least one chemical and/or physical change resulting from an interaction between the TMNP matrix and the analyte molecules in the sample. The active surface may be a metal body or a metallic surface of a metal selected from gold, platinum, silver, and alloys thereof. In some embodiments, the active surface is a non-metallic body, such as graphite, Indium-Tin-Oxide (ITO), glass and others, which may or may not be coated with a metallic coating.

In some embodiments, the active surface is an electrode. In other embodiments, the active surface is a metal (or alloy) coated glass.

The active surface may be a two-dimensional surface on top of which the matrix is formed or may be a three-dimensional body having, e.g., a circumference which is fully or partially associated with the matrix. In some embodiments, the matrix completely covers the active surface. In other embodiments, the matrix is formed on spaced-apart regions of the active surface.

In some embodiments, the matrix is associated with said active surface through one or more surface-binding moieties. The surface-binding moieties may or may not be the same as the recognition groups used to associate the plurality of TMNP in the matrix. In some embodiments, where the active surface is a gold surface and the TMNPs are gold nanoparticles, the surface-binding moieties and the recognition groups compose sulfur containing groups, such as thiols, as further disclosed hereinabove. In further embodiments, the surface-binding moieties and the recognition groups are p-thioaniline or a dimer or oligomer thereof.

In order to associate the matrix with the active surface, it is not necessary to have all nanoparticles of the matrix associated with the surface. It is merely required that a portion of the matrix is associated with the surface through the surface-binding groups.

It should be noted, that in embodiments where electropolymerization is employed for the construction of the matrix, the matrix may contain electropolymerized recognition groups and electropolymerized surface-binding groups of various lengths (a varying number of monomers, e.g., p-thioaniline monomers). For example, the matrix may be composed of nanoparticles which are associated with each other via dimers of p-thioaniline and nanoparticles which are associated via a different oligomer, e.g., trimer, quartermer, etc. Thus, in some embodiments, the matrix is inhomogeneous, i.e., not arranged from a single type of recognition group nor is it arranged in an ordered multilayered structure.

The analyte molecules which may be detected, using a method according to the invention, are numerous. As the matrix, i.e., TMNPs and recognition groups may be tailored to assay the presence and/or quantity of a certain analyte in a sample, the method of the invention may be both generic and, as desired, analyte-specific. In some embodiments, the analyte to be assayed is an organic material. In other embodiments, the analyte is a nitro-bearing compound, e.g., an aromatic nitro compound. In still other embodiments, the analyte is selected from nitrotoluenes (ortho-, meta- and para-), dinitrotoluenes (all isomers, e.g., 2,3-, 2,4-, 2,5-, 2,6-, 3,4-, 3,5-dinitrotoluene), trinitrotoluenes (all isomers, e.g., 2,3,4-, 2,3,5- 2,3,6-, 2,4,5-, 2,4,6-, 3,4,5-trinitrotoluene), nitrophenol (ortho-, meta- and para-), dinitrophenoles (all isomers, e.g., 2,3-, 2,4-, 2,5-, 2,6-, 3,4-, 3,5-dinitrophenol) and trinitrophenoles (all isomers, e.g., 2,3,4-, 2,3,5- 2,3,6-, 2,4,5-, 2,4,6-, 3,4,5-trinitrophenol). In some further embodiments, the analyte is a trinitrotoluene or trinitrophenol, e.g., 2,4,6-trinitrotoluene (TNT), 2,4,6-trinitrophenol (picric acid) or a combination thereof.

The method of the invention may be carried out by bringing into contact the matrix comprising the TMNPs, as defined herein, with a sample (control or the so-called field-sample suspected of comprising the analyte) in such a way to permit interaction between the recognition groups of the matrix and the analyte molecules. For achieving interaction, the matrix may be introduced into the sample (e.g., by dipping) for a period of time sufficient to achieve (not necessarily complete) interaction. The dipping may be repeated. Alternatively, the sample may be added onto the matrix (e.g., dripping). Other methods are suitable alternatives. Typically, the matrix and the sample are brought into contact at room temperature.

The interaction between the matrix, i.e., the recognition groups, and the analyte molecules, e.g., TNT molecules, may be probed by monitoring at least one measurable change, the change being associated with a change in at least one property or structure of the target molecule or one or more component of the matrix (or the matrix as a whole) caused by said interactions. Specifically, the measurable change may be, for example, in any one electric property or any one electrochemical property or any one spectroscopic property.

In some embodiments, the at least one change is in at least one electric property of the analyte and/or the matrix. The change may be measured by determining, e.g., current-voltage relationship, impedance, and other parameters, prior to and after the matrix and the sample have been brought into contact with each other. For quantitative measurements, calibration curves may be used.

In some other embodiments, the at least one change is in at least one optical property of the analyte and/or the matrix. Such optical property may be detected by Surface Plasmon Resonance (SPR), infra-red (IR) spectroscopy, Raman spectroscopy, X-ray photoelectron spectroscopy (XPS), photonic detection, evanescent detection, and cantilever detection.

In some embodiments, the qualitative and/or quantitative analysis of the analyte is achieved by employing SPR to probe a change in a dielectric property of the analyte and/or the matrix or a combination thereof. In some embodiments, for SPR measurements, the active surface to which the matrix is bound is a gold-coated glass, e.g., an SPR cell (chip).

Thus, in some embodiments, the method of the invention comprises:

(a) providing nanoparticles of a transition metal, said nanoparticles carrying a plurality of recognition groups capable of undergoing interaction with analyte molecules, e.g., TNT molecules;

(b) contacting said nanoparticles with a sample suspected of containing analyte molecules;

(c) providing assay conditions to permit interaction between said recognition groups and the analyte molecule(s), e.g., TNT molecules; and

(d) probing the interaction to thereby detect at least one change in at least one dielectric property in the vicinity of the nanoparticles, whereby said change is indicative of at least the presence and quantity of said analyte molecule(s), e.g., TNT molecules, in the sample.

In another aspect the invention provides an electrode for carrying the method of the invention. In some embodiments, the electrode has a conductive surface connected to a matrix, said matrix comprising a plurality of transition metal nanoparticles (TMNPs), wherein substantially each of said nanoparticles is connected to another by at least one recognition group capable of mediating electron transfer between nanoparticles of the matrix; at least a portion of said plurality of nanoparticles is connected to said conductive surface by at least one surface binding group, capable of mediating electron transfer between the matrix and said conductive surface.

In some embodiments, each of the TMNPs is selected as defined above.

In some embodiments, the matrix is produced by molecular imprinting.

Thus, the invention also provides a method for molecular imprinting of a matrix for detecting an analyte, said method comprising:

modifying the surface of a solid support through the attachment of functional to groups, e.g., the surface binding groups,

reacting, in the presence of at least one guest molecule, the functional groups of the modified solid support with transition metal nanoparticles carrying a plurality of recognition groups capable of undergoing interaction with analyte molecules, under conditions allowing formation of a matrix embedded with said at least one guest molecule,

wherein said matrix is thereby composed of a plurality of nanoparticles associated with each other through recognition groups; and

removing said at least one guest molecule to thereby produce a molecularly-imprinted matrix on the solid substrate.

Without wishing to be bound by theory, the imprinting method increases, together with the complementary π-donor-acceptor interactions, the association of the analyte molecules, e.g., TNT, to the sensing electrode (active surface of the solid support), thereby enhancing the sensitivity of the analysis.

In some embodiments, the at least one guest molecule is selected to have at least one of shape, size, substitution and electronic structure and distribution as that of the analyte molecule to be detected. In some embodiments, the at least one guest molecule is identical to the analyte molecule. In some further embodiments, the at least one guest molecule has the same substituents and substituent pattern as the analyte molecule. In further embodiments, the at least one guest molecule is larger in its overall space occupying volume than that of the analyte molecule. In additional embodiments, the at least one guest molecule is a mixture of two or more guest molecules, one of which may or may not be the same as the analyte molecule. For TNT detection, the guest molecule is selected from TNT and picric acid.

For the purpose of employing the matrix thus formed for assaying the presence and/or quantity of a certain analyte molecule, the imprinting method of the invention provides for the removal of the guest molecule from the matrix, to thereby form the analyte-recognition fields. The at least one guest molecule may be removed from the matrix in the imprinting process by contacting, e.g., washing the matrix with a suitable solvent, such as an organic solvent or an aqueous solution at a desired pH. In some embodiments, the washing solution is an aqueous solution or a buffer at a substantially neutral pH (˜6.5-7.5). In some embodiments, the buffer used has an acidic or basic pH.

In some embodiments, the method of imprinting further comprises the step of verifying the total removal of the guest molecules.

The method may further comprise the step of determining the base-line property of the matrix to be used in the calibration of the matrix or device. The base-line property is typically identical to the electric, electrochemical and/or optical property used to probe the change in the matrix after contact with the analyte sample. For example, if SPR measurements are used to assay the presence of TNT molecules in a sample, the dielectric properties of the matrix prior to coming in contact with the sample will be determined as the base-line property of the matrix.

In some embodiments, the solid support is an electrode or a coated glass slide (cell or chip). In some further embodiments, the glass cell is coated with gold.

In some embodiments, the matrix is formed by electropolymerization.

The matrix produced by the imprinting method of the invention, may be used in a method for detecting an analyte, e.g., TNT, using the molecularly-imprinted matrix, the method comprising exposing the molecularly-imprinted matrix to a sample suspected of containing said analyte and detecting the interaction of the analyte, as disclosed herein, with the matrix. It should be noted, that while the present invention discloses an imprinting method for the production of the matrix, the matrix may be produced by any other process provided that it follows the definition and characteristics provided herein.

In some embodiments, the interaction (physical or chemical) is detected using electric or optical methods, e.g., SPR or voltammetric measurements.

In a further aspect there is provided a device for carrying out the detection of an analyte in a sample, said device comprising at least one assay unit having a plurality of nanoparticles of a transition metal, said nanoparticles carrying recognition groups capable of undergoing interaction with the analyte molecule(s), under predetermined assay conditions. The device may further comprise means to probe the interaction between said recognition groups and the analyte molecule(s) and means for detecting at least one change in at least one measurable property (electric or optical). In some embodiments, where the device is intended for electric measurements, the assay unit may comprise an electrode. For optical analysis, the assay unit may, for example, be in the form of an SPR cell or chip.

The invention also provides a sensor comprising an electrode according to the invention.

### BRIEF DESCRIPTION OF THE DRAWINGS

In order to understand the invention and to see how it may be carried out in practice, embodiments will now be described, by way of non-limiting example only, with reference to the accompanying drawings, in which:

FIGS. 1A-1C are (A) Linear sweep voltammograms corresponding to the reduction of (a) 2.3, (b) 4.6, (c) 6.9, and (d) 9.2 ppm TNT in PB at a bare Au electrode. Scan rate is 20 mV s⁻¹. The scan direction is from positive to negative. (B) Linear sweep voltammograms corresponding to the reduction of (a) 17 ppb; (b) 34 ppb; (c) 72, (d) 144, (e) 288, (f) 575, and (g) 2300 ppb TNT in PB at a p-aminothiophenol-functionalized Au electrode. Scan rate is 20 mV s⁻¹. The scan direction is from positive to negative. (C) Calibration curves corresponding to the analysis of TNT at the (a) p-aminothiophenolfunctionalized Au electrode and (b) bare Au electrode. All data were recorded after interacting the respective electrodes with the TNT solution sample for a time interval of 50 s. All of the experiments were performed under an inert Ar atmosphere.

FIGS. 2A-2B are (A) Linear sweep voltammograms corresponding to the reduction of (a) 0.46, (b) 1.8, (c) 4.5, (d) 9, (e) 81, and (f) 143 ppb TNT in PB on an oligoaniline-cross-linked Au NPs-functionalized electrode. Scan rate is 20 mV s⁻¹. The scan direction is from positive to negative. (B) Calibration curves corresponding to the analysis of TNT at the (a) oligoaniline-crosslinked Au NPs-functionalized electrode and (b) p-aminothiophenol-functionalized Au electrode. All data were recorded after interacting the respective electrodes with the TNT solution sample for a time interval of 50 s. All of the experiments were performed under an inert Ar atmosphere.

FIG. 3 presents coulometric analysis of the TNT associated with the oligoanilinecross-linked Au NPs-functionalized electrode upon interaction of the electrode with different bulk concentrations of TNT. The functionalized electrode was immersed in the different solutions of TNT for 2 h.

FIGS. 4A-4B are (A) Calibration curves corresponding to the analysis of (a) 2,4-DNT and (b) 4-NT at the oligoaniline-cross-linked Au NPs-functionalized electrode. (B) Calibration curves corresponding to the analysis of (a) TNT and (b) 2,4-DNT at the oligoaniline-cross-linked Au NPs-functionalized electrode. All data were recorded after interacting the respective electrodes with the TNT solution sample for a time interval of 50 s. All of the experiments were performed under an inert Ar atmosphere.

FIGS. 5A-5B are (A) Calibration curves corresponding to the analysis of TNT at the (a) picric acid imprinted oligoaniline-cross-linked Au NPs-functionalized electrode and (b) nonimprinted oligoaniline-cross-linked Au NPs-functionalized electrode. All data were recorded after interacting the respective electrodes with the TNT solution sample for a time interval of 50 s. (B) Coulometric analysis of the TNT associated to the picric acid imprinted oligoaniline-cross-linked Au NPs-functionalized electrode upon interaction of the electrode with different bulk concentrations of TNT. The functionalized electrode was immersed in the different solutions of TNT for 2 h.

FIG. 6 presents time-dependent electrical responses upon the analysis of an aqueous TNT sample, 0.1 mM, by the (a) nonimprinted oligoaniline-bridged Au NPs functionalized electrode and (b) imprinted oligoaniline-bridged Au NPs-modified electrodes. In all experiments the scanned range was −0.4 to −0.6 V vs SCE. Scan rate was 25 mV s⁻¹. Experiments were performed under an inert Ar atmosphere.

FIG. 7 presents calibration curves corresponding to the analysis of (a) TNT and (b) 2,4-DNT by the picric acid imprinted oligoaniline-cross-linked Au NPs-functionalized electrode. All data were recorded after interacting the respective electrodes with the TNT solution sample for a time interval of 50 s. All of the experiments were performed under an inert Ar atmosphere.

FIGS. 8A-8C are (A) SPR curves corresponding to the bis-aniline AuNPs composite: (a) before the addition of trinitrotoluene, and, (b) after the addition of trinitrotoluene, 200 nM. Inset: SPR curves corresponding to a bilayer of AuNPs crosslinked by 1,4-butane dithiol and linked to the Au surface: (a) before, and, (b) after the addition of trinitrotoluene, 200 nM. (B) Sensogram corresponding to the changes in the reflectance intensities, at θ=62.4°, upon addition of variable concentrations of trinitrotoluene: (a) 10 pM, (b) 20 pM, (c) 40 pM, (d) 100 pM, (e) 1 nM, (f) 10 nM, (g) 50 nM, (h) 200 nM, (i) 1 μM, (j) 5 μM. (C) Calibration curve relating the reflectance changes to the concentration of trinitrotoluene. All measurements were performed in a 0.1 M HEPES buffer solution, pH=7.2.

FIGS. 9A-9B are (A) SPR curves corresponding to the picric acid-imprinted bis-aniline AuNPs composite: (a) before the addition of trinitrotoluene, and (b) after the addition of trinitrotoluene, 1 pM. Inset: SPR curves (enlarged at the minimum reflectance angle) corresponding to the picric acid-imprinted bis-aniline AuNPs composite in the presence of different concentrations of trinitrotoluene. (B) Sensogram corresponding to the changes in the reflectance intensities, at θ=62.4°, upon addition of variable concentrations of trinitrotoluene: (a) 10 fM, (b) 20 fM, (c) 50 fM, (d) 100 fM, (e) 1 pM, (f) 5 pM. All measurements were performed in a 0.1 M HEPES buffer solution, pH=7.2.

FIG. 10A presents the calibration curve relating the reflectance changes to the concentration of trinitrotoluene.

FIG. 10B presents the changes in the reflectance intensities upon analyzing trinitrotoluene within a broad range of concentrations (presented on a semilogarithmic scale): (a) The imprinted bis-aniline-crosslinked AuNPs composite. (b) The non-imprinted bis-aniline-crosslinked AuNPs composite. All measurements were performed in a 0.1 M HEPES buffer solution, pH=7.2.

FIGS. 11A-11B are (A) Calibration curves (presented on a semilogarithmic scale) corresponding to the analysis of: (a) trinitrotoluene, and (b) 2,4-dinitrotoluene by the imprinted bis-aniline-crosslinked AuNPs composite. (B) Calibration curves (presented on a semilogarithmic scale) corresponding to the analysis of: (a) 2,4-dinitrotoluene, and (b) 4-nitrotoluene, by the imprinted bis-aniline-crosslinked AuNPs composite. All measurements were performed in a 0.1 M HEPES buffer solution, pH=7.2.

FIG. 12 presents the effect of applied potential on the reflectance changes, at θ=62.4°, of the bis-anilinecrosslinked AuNPs composite, upon analyzing trinitrotoluene, 100 fM: (a) The imprinted composite, and (b) The non-imprinted composite. All measurements were performed in a 0.1 M HEPES buffer solution, pH=7.2.

FIGS. 13A-13B are (A) Calculated changes in the real-part of the dielectric constant of the imprinted bis-anilinecrosslinked AuNPs composite upon analyzing different concentrations of trinitrotoluene. (B) Calculated changes in the imaginary-part of the dielectric constant of the imprinted bis-aniline-crosslinked AuNPs composite upon analyzing different concentrations of trinitrotoluene.

FIG. 14 shows the calculated conductivity of the imprinted bis-aniline-crosslinked AuNPs composite upon analyzing different concentrations of trinitrotoluene.

### DETAILED DESCRIPTION OF EMBODIMENTS

Electrochemical sensors for the analysis of TNT with enhanced sensitivities are herein disclosed. The enhanced sensitivities are achieved by tailoring π-donor-acceptor interactions between TNT and π-donor modified electrodes or π-donor-cross-linked Au nanoparticles linked to the electrode. In one configuration a p-aminothiophenolate monolayer-modified electrode leads to the analysis of TNT with a sensitivity corresponding to 17 ppb (74 nM). In the second configuration, the cross-linking of Au NPs by oligothioaniline bridges to the electrode yields a functionalized electrode that detects TNT with a sensitivity that corresponds to 460 ppt (2 nM). Most impressively, the imprinting of molecular TNT recognition sites into the π-donor oligoaniline-cross-linked Au nanoparticles yields a functionalized electrode with a sensitivity that corresponds to 46 ppt (200 pM). The electrode reveals high selectivity, reusability, and stability.

### Nanoparticles Synthesis

Gold nanoparticles functionalized with 2-mercaptoethane sulfonic acid and p-aminothiophenol (Au NPs) were prepared by mixing a 10 mL solution containing 197 mg of HAuCl4 in ethanol and a 5 mL solution containing 42 mg of mercaptoethane sulfonate and 8 mg of p-aminothiophenol in methanol. The two solutions were stirred in the presence of 2.5 mL of glacial acetic acid in an ice bath for 1 h. Subsequently, 7.5 mL of aqueous solution of 1 M sodium borohydride, NaBH₄, was added dropwise, resulting in a dark color solution associated with the presence of the Au NPs. The solution was stirred for 1 additional hour in an ice bath and then for 14 h at room temperature. The particles were successively washed and centrifuged (twice in each solvent) with methanol, ethanol, and diethyl ether. An average particle size of 3.5 nm was estimated using TEM. Nanopure (Barnstead) ultrapure water was used in the preparation of the different solutions. Au-coated glass plates (Evaporated Coatings, PA, USA) were used as working electrodes. Prior to modification, the Au surface was flame-annealed for 5 min in an n-butane flame and was allowed to cool down for 10 min under a stream of Ar. Cyclic voltammetry experiments were carried out using a PC-controlled (Autolab GPES software) electrochemical analyzer potentiostat/galvanostat (μAutolab, type III). A graphite rod (d=5 mm) was used as a counter electrode, and the reference was a saturated calomel electrode (SCE).

### Chemical Modification of the Electrodes

p-Aminothiophenol-functionalized electrodes were prepared by immersing the Au plates for 24 h into a p-aminothiophenol ethanolic solution, 50 mM. In order to prepare the oligoaniline Au-NPs film on the electrode, the surface-tethered p-aminothiophenol groups were electropolymerized in the presence of the p-aminothiophenol-functionalized Au NPs in an electrolyte solution of 0.1 M phosphate buffer (PB) pH=7.4 that contained 1 mg·mL⁻¹ of the NPs. The polymerization was performed by the application of six potential cycles between −0.35 and 0.5 V, at a potential scan rate of 100 mV s-1. The resulting films were washed with the background electrolyte solution to exclude any residual monomer from the electrode. Similarly, picric acid imprinted oligoaniline films were prepared by adding 1 mg·mL-1 picric acid to the Au NPs mixture prior to the electropolymerization process. The extraction of the picric acid from the film was carried out by immersing the electrodes in a 0.1 M phosphate buffer solution, pH=7.4 for 2 h at room temperature under continuous agitation. The full removal of picric acid from the electropolymerized film was verified electrochemically. Prior to the sensing experiments, the PB solutions were loaded with the analytes and purged for 5 min with N2. The current values for analyzing TNT in the different systems were derived by subtracting the current generated by the pure buffer solution from the peak current of the voltammetric wave at ca. −0.5 V vs SCE.

### Chemical Modification for SPR

p-Aminothiophenol-functionalized electrodes were prepared by immersing the Au slides for 24 hours into a p-aminothiophenol ethanolic solution, 50 mM. In order to prepare the bis-aniline-crosslinked AuNPs composite on the electrode, the surface-tethered p-aminothiophenol groups were electropolymerized in a 0.1 M HEPES buffer solution (pH=7.2) containing 1 mgml⁻¹ of p-aminothiophenol-functionalized AuNPs. The polymerization was performed by the application of 10 potential cycles between −0.35 and 0.8 V vs. Ag wire quasi-reference electrode, at a potential scan rate of 100 mVs⁻¹, followed by applying a fixed potential of 0.8 V for 30 minutes. The resulting films were, then, washed with the background buffer solution to exclude any residual monomer from the electrode. Similarly, picric acid-imprinted oligoaniline-crosslinked films were prepared by adding 1 mgml⁻¹ picric acid to the AuNPs mixture prior to the electropolymerization process. The extraction of the picric acid from the film was carried out by immersing the electrodes in a 0.1 M HEPES solution, pH=7.2 for 2 hours at room temperature. The full removal of picric acid from the electropolymerized film was verified electrochemically. In a control experiment, a two-layer AuNPs matrix was assembled on the Au-coated glass surface by the primary association of thiopropionic acid-stabilized AuNPs (diameter 3.5 nm) on the cystaminemonolayer-functionalized gold slide, followed by the assembly of a second AuNPs layer by crosslinking the second layer of AuNPs to the base AuNPs layer using 1,4-butane dithiol.

### Surface Plasmon Resonance Instrumentation

A surface plasmon resonance (SPR) Kretschmann type spectrometer NanoSPR 321 (NanoSPR devices, USA), with a LED light source, λ=650 nm, and with a prism refraction index of n=1.61, was used. The SPR sensograms (time-dependent reflectance changes at a constant angle) represent real-time changes and these were measured in situ using a home-built fluid cell. Au-coated semi-transparent glass slides (Mivitec GmbH, Analytical μ-Systems, Germany) were used for the SPR measurements. Prior to modification, the Au surface was cleaned in a hot ethanol, at 60° C., for 30 min. For the electrochemical polymerization and SPR measurements employing in situ constant potential application, an auxiliary Pt (0.5 mm diameter wire) and a quasi-reversible reference Ag electrode (QRE) (0.5 mm diameter wire) were installed into a Perspex cell (volume 0.5 cm³, working area 0.4 cm²). For the constant potential measurements the SPR electrode potential was biased (vs. Ag QRE), and the respective SPR curve was recorded in the presence of 100 fM trinitrotoluene. For these measurements an Autolab electrochemical system (Echo Chemie, The Netherlands) driven by GPES software was used. Atomic force microscopy (AFM) images were captured in a tapping mode on a Digital Nanoscope IV instrument employing Si cantilevers (NSC15/AIBS, MicroMasn, Estonia, resonance frequency order of 320 kHz).

### Fitting of Experimental Results

Fresnel's equation-based SPR modeling for a five-layer system was performed using Winspall 2.0 program, generously provided by Prof. W. Knoll (Max Plank Institute for Polymer Research in Mainz, Germany). A refractive index for bulk Au, n=0.173+3.422i, was used for the modeling as the refractive index for the AuNPs. The Langmuir isotherm fittings were performed using Origin 7.5 software (Origin Lab Corporation).

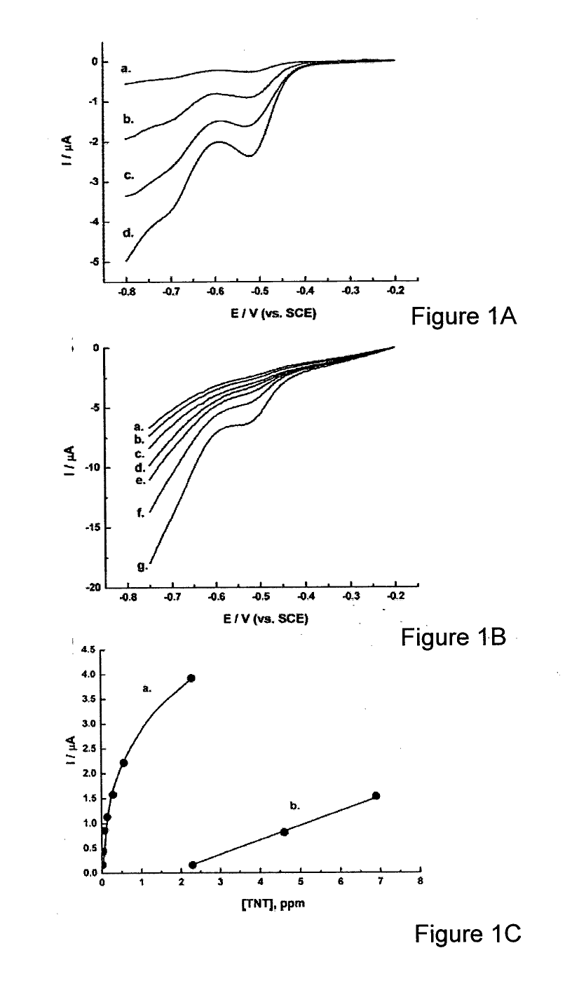

Nitrobenzene units undergo stepwise reduction to hydroxylamine groups according to Eq. 1. FIG. 1A depicts the linear sweep voltammograms of TNT at a bare Au electrode. The lowest level of TNT that is detectable at the bare Au electrode is 2.3 ppm (10 μM). The aim of the present invention was to enhance the sensitivity of TNT analysis by modifying the electrode surface with π-donor groups that would concentrate the TNT analyte at the electrode surface by π-donor-acceptor interactions. Thus, the Au surface was modified with p-aminothiophenol that acts as a π-donor, Scheme 1A.

FIG. 1B shows the cyclic voltammograms of variable concentrations of TNT at the 2-functionalized Au electrode. The amperometric responses of the electrode are observed at substantially lower bulk concentrations of TNT, as compared to the bare Au electrode. FIG. 1C shows the derived calibration curves that correspond to the analysis of TNT at the 2-modified electrode, curve (a), and at the bare Au surface, curve (b). The TNT can be detected at the p-aminothiophenol-functionalized surface with a sensitivity that corresponds to 17 ppb (74 nM). The 135-fold increase in the sensitivity for analyzing TNT by the 2-functionalized electrode is attributed to the concentration of the analyte at the electrode surface by π donor-acceptor interactions with the monolayer modifier.

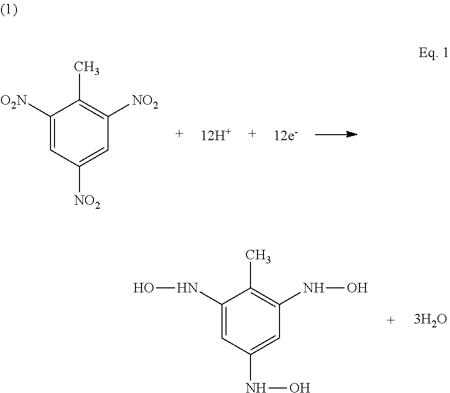

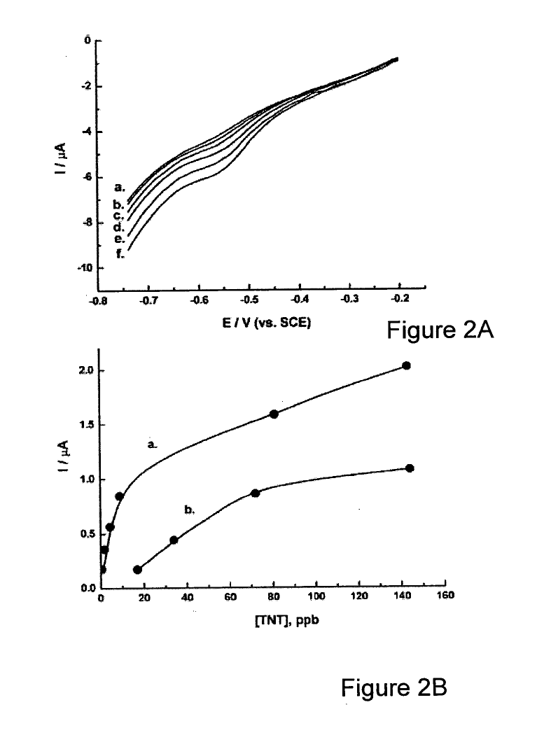

To further enhance the sensitivity of the detection of TNT, p-aminothiophenol-functionalized Au nanoparticles were employed as co-modifier of the Au electrode, Scheme 1B. Au NPs, 3.5 nm in diameter, were prepared by the sodium borohydride reduction method, and the particles were modified with a mixed capping monolayer consisting of polymerizable p-aminothiophenolate and 2-mercaptoethane sulfonic acid. The latter component enhances the solubility of the NPs in the aqueous medium. The functionalized Au NPs were then electropolymerized in the presence of the 2a-functionalized Au electrode to yield the oligoaniline π-donor-bridged Au NP aggregates on the electrode surface. Without wishing to be bound by theory, enhanced sensitivity for analyzing TNT at the resulting Au NP-functionalized electrode is due to two complementary effects: (i) The content of the π-donor oligoaniline units increases as a result of the formation of Au NP aggregates; (ii) the Au NPs provide a conductive roughened array, and thus, electrochemical analysis of TNT is feasible at a roughened surface with a higher content of π-donor sites for the given concentration of the analyte. FIG. 2A shows the linear sweep voltammograms observed upon analyzing variable concentrations of TNT by the Au NPsfunctionalized electrode. FIG. 2B, curve (a) shows the derived calibration curve. By applying the electrochemically aggregated π-donor Au NPs electrode, the TNT is sensed with a detection limit that corresponds to 460 ppt (2 nM). For comparison, FIG. 2B, curve (b) depicts the calibration curve for analyzing TNT by the polymerizable p-aminothiophenolate-monolayer-functionalized electrode. The amperometric responses in the presence of the Au NP-modified electrode, in the lower concentration range of TNT, are substantially higher, and the sensitivity is improved by a factor of 37 compared with the monolayer configuration. The sensitivity observed with the Au NP-modified electrode is impressive, and hence this electrode was structurally and functionally characterized.

The covalent binding of the Au NP bridged to the electrode was followed by quartz crystal microbalance experiments. Upon the electropolymerization of the polymerizable p-aminothiophenolate-functionalized Au NPs onto the Au/quartz crystal, a frequency decrease that corresponded to 300 Hz was observed. This value translates to a surface coverage of the particles that corresponds to ca. 3×10¹² Au NPs·cm⁻².

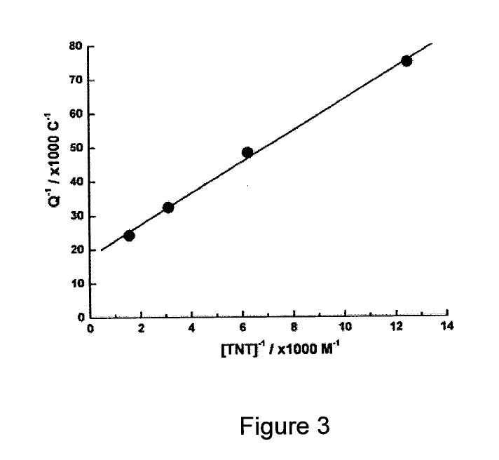

The association constant of TNT to the oligoaniline π-donor bridging units associated with the Au NPs, Eq. 2, was determined electrochemically. The association constant is given by Eq. 3, where R is the number of π-donor sites in the system and θ is the fraction of sites that is complexed by TNT at any bulk concentration of the analyte. Eq. 3 can be rewritten in the form of Eq. 3a, and the value of θ is derived from the coulometric analysis of the first wave of reduction of TNT at any bulk concentration of TNT. The charge associated with the bound TNT is proportional to the number of occupied π-donor sites. FIG. 3 shows the analysis of the association constant of TNT to the binding sites according to Eq. 3a. The derived association constant corresponds to Ka=3100±50 M⁻¹. It should be noted that this method for deriving the association constants assumes that the binding of TNT to the π-donor sites is unaffected by neighboring occupied sites. At the low concentrations of TNT at which the association constant was derived, this assumption is justified.

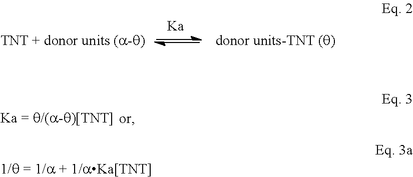

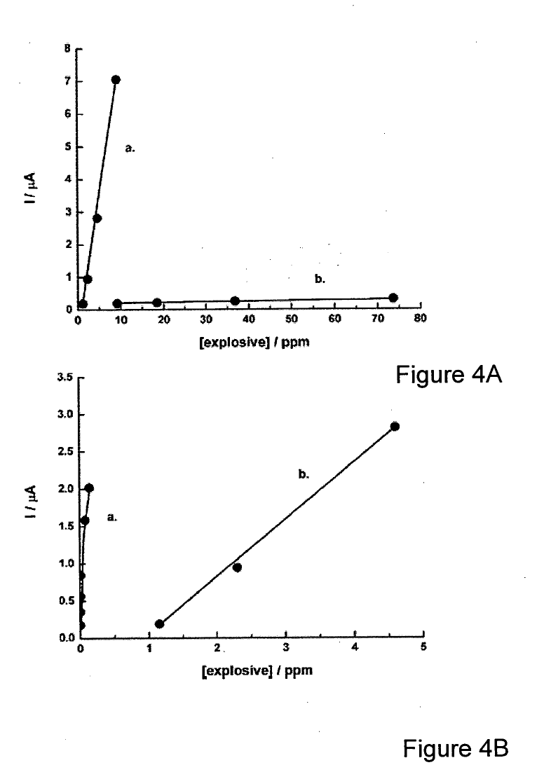

An important aspect of the Au NPs-functionalized electrodes relates to their specificity toward detecting different nitrotoluene explosives. Accordingly, the sensing of 2,4-dinitrotoluene, DNT, and of 4-nitrotoluene, NT, by the Au NPs-functionalized electrode was examined. FIG. 4A, curves (a) and (b), depicts the resulting calibration curves. The detection limits for analyzing DNT and NT correspond to 1.1 ppm (5 μM) and 9.2 ppm (40 μM), respectively. Evidently, these values are 2.6×10³-fold and 2.0×10⁴-fold lower than the sensitivity for the detection of TNT. These results are consistent with the fact that DNT and NT exhibit lower π-acceptor properties due to the decreased number of the electron-withdrawing nitro groups, and hence their concentration at the electrode surface via π-donoracceptor interactions is substantially lower. It is therefore expected that the sensitivities for the detection of the nitroaromatic substrates decrease as the π-acceptor properties of the analytes are lowered. FIG. 4B compares the calibration curves for analyzing TNT and DNT by the Au NPs functionalized electrode. A selectivity factor of ca. 20 (corresponding to the ratio of the slopes) is derived. The Au NP-modified electrode can be recycled by extracting the analyte TNT, and it reveals an excellent stability (extraction of the bound TNT was performed by shaking the electrode in a phosphate buffer solution, pH=7.4, for 2 h). The Au NPs-modified electrode was operated for seven days with no noticeable change in its functional activity. It should be noted that TNT and DNT reveal no selectivity upon electrochemical analysis by the same Au electrode. These results imply that the selectivity is, indeed, induced by the π-donor-acceptor interactions between the different nitroaromatic compounds and the π-donor oligoaniline bridges.

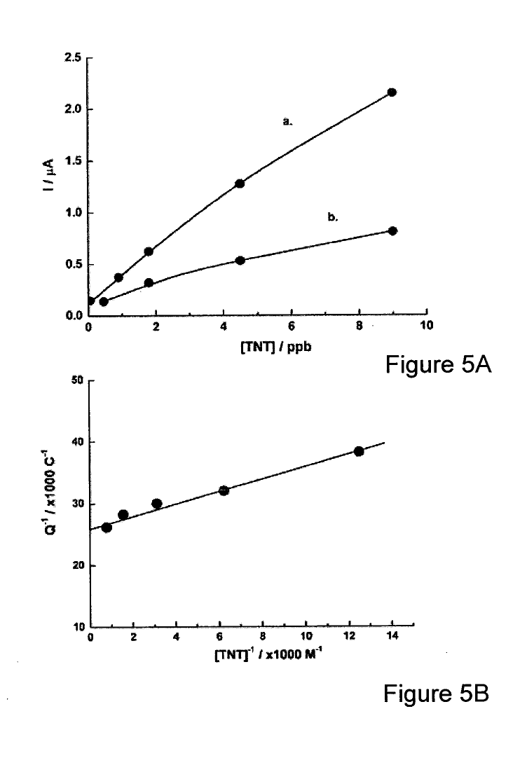

Although the Au NPs-functionalized electrode revealed an impressive sensitivity, we searched for possibilities to enhance the sensitivity (as well as the selectivity) of the electrode for analyzing TNT. This could possibly be accomplished by increasing the binding affinity of the analyzed substrate to the Au NP sensing surface. Toward this end, we realized that the imprinting of molecular recognition sites for TNT in the oligoaniline π-donor bridged Au NPs array associated with the electrode might provide an effective means for enhancing the sensitivity of the sensor electrode. That is, in addition to the association of TNT to the π-donor sites, the formation of imprinted π-donor molecular contours around the complex might synergistically bind the TNT analyte to the sensing surface, thus increasing the association constant. Accordingly, picric acid (6) was used as the imprinting substrate. The imprint molecule has three nitro groups, analogous to the analyte TNT, the OH functionality resembles the dimensions of the methyl group, and the molecule exhibits strong π-acceptor properties. The high solubility of NT in water permits the effective formation of the π-donor acceptor complex between 6 and the polymerizable p-aminothiophenolate-functionalized Au NPs, Scheme 2. Accordingly, the picric acid complexed polymerizable p-aminothiophenolate-functionalized Au NPs were electropolymerized at the polymerizable p-aminothiophenolate-modified electrode, and the imprint molecules of picric acid were then removed by extraction to yield the imprinted Au NPsfunctionalized electrode, Scheme 2. The resulting electrode was then used to analyze TNT. FIG. 5A, curve (a) shows the calibration curve that corresponds to the analysis of TNT by the NT-imprinted oligoaniline-bridged Au Nps electrode. For comparison, curve (b) depicts the calibration curve observed with the nonimprinted cross-linked Au NPs electrodes. Evidently, the amperometric responses with the imprinted Au NPs electrode are substantially higher as compared to the nonimprinted electrode, within a similar concentration range of the analyte. The sensitivity for analyzing TNT by the NT-imprinted Au NPs electrode corresponds to 46 ppt (200 pM), a value that is 10-fold higher than the sensitivity with the nonimprinted Au NPs electrode (and 5×10⁴ fold higher than the initial, bare Au electrode configuration). To account for the enhanced sensitivity observed with the imprinted Au NPs array, we analyzed the association constant of TNT to the imprinted sensing surface. FIG. 5B shows the coulometric analysis of the TNT bound to the imprinted Au NPs electrode, at different bulk concentrations of TNT, according to eq 3a. The derived association constant corresponded to (2.6×10⁴)±600 M⁻¹, a value that is ca. 8-fold higher than that with the nonimprinted oligoaniline bridged Au NPs electrode. Thus, the enhanced sensitivity for analyzing TNT by the imprinted electrode is attributed to improved concentration of the analyte at the electrode surface as a result of higher affinity of TNT to the imprinted π-donor sites. The imprinted oligoaniline-cross-linked Au NP modified electrode operated for 1 week at room temperature, showing a ca. 10% decrease in the TNT signal.

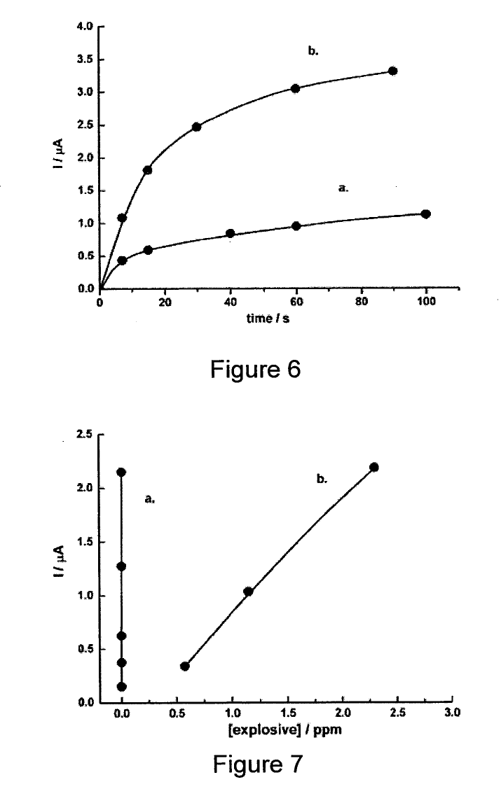

Throughout the study, the sensing electrodes were interacted with the TNT samples for a time interval of 50 s, prior to the electrochemical probing of the TNT signals. This time interval was selected after a detailed analysis of the kinetics of TNT binding to the imprinted and nonimprinted oligoaniline-bridged aggregates, associated with the electrodes. FIG. 6, curve (a) depicts the kinetics of association of TNT, 0.1 μM, to the nonimprinted Au NPs-functionalized electrode, whereas curve (b) shows the electrical response of the imprinted, oligoaniline-bridged Au NPs-functionalized Au electrode. After ca. 50 s, the electrical response of the imprinted electrode tends to level off. We found that, within the entire concentration range for analyzing TNT, a time interval of 50 s for incubating the functionalized electrodes with the samples was sufficient for generating an electrical response corresponding to 85-95% of the saturation value. These results clearly imply that the response of our sensor device is rapid and, thus, of potential practical applicability.

Furthermore, for any practical use, the analysis of TNT in real environmental samples should be elucidated. Thus, we applied the imprinted Au NPs-functionalized electrodes for analyzing aqueous groundwater and seawater samples contaminated with variable concentrations of TNT. The results indicated that the electrical responses of TNT in the different media showed similarity, within ±12%, to the results obtained in pure buffer solution.

To complete the study, the selectivity features of the imprinted Au NPs electrode were analyzed and compared to the selectivity pattern of the nonimprinted electrode. FIG. 7, curve (a) depicts the calibration curve that corresponds to the analysis of TNT by the imprinted Au NPs electrode, whereas curve (b) shows the calibration curve that corresponds to the analysis of dinitrotoluene, DNT, at the imprinted Au NPs electrode. The selectivity factor (βTNT/βDNT), where β is the slope of the respective calibration curve, equals 215. This selectivity factor is ca. 11-fold higher than the selectivity observed for the nonimprinted Au NPs electrode. Thus, the imprinting procedure of picric acid not only increases the sensitivity of the modified electrode but also impressively enhances its selectivity toward the analysis of TNT.

It should be noted that several previous studies used particles for the analysis of imprinted analytes. For example, imprinted core-shell silica particles were used for sensing TNT [28] Similarly, imprinted photonic polymers were used for chiral recognition [29]. The imprinting method in the present study is, however, completely different than the reported methodologies [28, 29]. While the previous studies used traditional imprinting procedures in organic or inorganic polymer matrices and focused on miniaturizing the polymer sizes into small beads, our imprinting approach is entirely different and may be considered as “imprinting at the nanoscale”. In our system, the functionalized Au NPs act as the “monomer units” for the electropolymerization imprinting process.

As the above clearly indicates, the modified electrodes of the invention are useful in the ultrasensitive detection of TNT by electrochemical means. As demonstrated, the modification of Au electrodes by a π-donor thioaniline monolayer improved the sensitivity for analyzing TNT by a factor of 135 as compared to a bare Au electrode. The electrochemical aggregation of Au NPs bridged by oligoaniline units on the Au electrode further increased the sensitivity of the modified electrode by a factor of 37 as a result of the formation of a high content of π-donor sites on the electrode surface and due to the three-dimensional conductivity of the NPs matrix. Finally, the imprint of molecular recognition sites into the π-donor oligoaniline-cross-linked Au NPs structure further enhanced the sensitivity by a factor of 10, and TNT was analyzed with a sensitivity that corresponded to 46 ppt (200 pM). Table 1 summarizes the detection limits for analyzing TNT in aqueous media by different sensor systems. It is evident that the imprinted π-donor Au NPs cross-linked array presents a highly sensitive method for analyzing TNT. The closest sensor configuration with comparable sensitivity involves immunosensor and SPR readout. The limited stability of antibodies and the long detection time intervals required by the TNT-induced displacement of the antibody from the SPR transducer highlight, however, the advantages of the present sensor system.

The successful imprinting of molecular recognition sites in aggregated structures of modified NPs should also be emphasized. This represents the first attempt to use modified particles to generate imprinted molecular sites. The use of metallic NPs, particularly Au NPs, to fabricate the imprinted sites, has important implications for future development of sensing devices. The three-dimensional conductivity of the Au NPs provides a means for the electrochemical readout of binding the analyte to the imprinted π-donor sites through the entire sensing surface. The formation of imprinted Au NP clusters may then be used to develop various new optical sensors. Furthermore, the present study used electropolymerization as a method to fabricate the functionalized imprinted Au NPs sensing matrix. Other methods, such as layer-by-layer deposition of Au NPs, may be similarly used to construct imprinted sites for improved sensing.

TABLE 1
Analysis of TNT in Aqueous Media by Different Sensor Systems

| | detection | | |
|---|---|---|---|
| Method | limit | | reference |
| imprinted electropolymerized | 46 | ppt | — |
| oligoanilinecross-linked Au NPs | | | |
| electrochemical determination by metallic | 1 | ppb | 5c |
| nanoparticle-carbon nanotube composites | | | |
| electrochemical detection by carbon nanotubes | 5 | ppb | 5d |
| electrochemical detection by mesoporous SiO2- | 414 | ppt | 6 |
| modified electrodes | | | |
| luminiscent oligo(tetraphenyl)silole | 20 | ppb | 3b |
| nanoparticles as chemical sensors | | | |
| remote microelectrode electrochemical sensor | 50 | ppb | 5a |
| in water | | | |
| adsorptive stripping detection by carbon | 600 | ppt | 5b |
| nanotube-modified GCE | | | |
| biochip (on gold) QCM detection | 1-10 | ppb | 7 |
| biochip (on gold) SPR detection | 1 | ppb | 7 |
| SPR immunosensor detection | 90 | ppt | 8 |

For the SPR studies, a similarly bisaniline-crosslinked AuNPs matrix was employed in association with an Au-coated glass surface. The formation of the π-donor-acceptor complexes between the nitro compound and the bis-aniline bridging units was, then, probed by following the effect of these complexes on the plasmon coupling of the AuNPs matrix to the surface plasmon wave. The effect of molecular imprinting of nitro-compound recognition sites into the bisaniline-crosslinked AuNPs composite on the sensitivity and selectivity of the resulting sensing surface is herein disclosed.

The bis-aniline-crosslinked AuNPs matrix was characterized by means of electrochemical, AFM and SPR measurements. Furthermore, by fitting of the experimental SPR curves, the dielectric functions of the sensing matrix upon analyzing the analyte are extracted. As generally noted above, even though the examples provided herein are specific to the application of the bis-aniline-crosslinked AuNP composite for the specific analysis of trinitrotoluene and picric acid, other applications of the method of the invention for the detection of other analytes may be considered.

Thioaniline-functionalized AuNPs, mean diameter 3.5 nm, were prepared by the capping of the AuNPs with a mixed monolayer of thioaniline and mercaptoethane sulfonate. The resulting functionalized AuNPs were electropolymerized on a thioaniline monolayer-modified flat Au electrode (a glass plate coated with a Au layer ca. 50 nm), by applying 10 electropolymerization cycles ranging between 0.80V to −0.35V vs. Ag/QRE, as depicted in Scheme 1(A). The thickness of the resulting matrix was estimated by ellipsometry measurements to be 9.3±1.7 nm with a volume fraction of AuNPs that equals to approximately 64%. This translates to the formation of an AuNPs composite consisting of an average number of approximately three layers (vide infra). The bis-aniline crosslinking units exhibit a quasireversible redox wave at 0.2 V vs. Ag/QRE. Coulometric analysis of the anodic peak corresponding to the bis-aniline units indicated a charge of 6×10₋₅ C·cm⁻² that translates to a total surface coverage of ca. 3.8×10¹⁴ aniline molecules·cm⁻².

The aniline units and bis-aniline bridging units exhibit π-donor properties and, thus, bind the trinitrotoluene electron acceptor by π-donor-acceptor interactions. The formation of the charge-transfer complex between the bis-aniline π-donor and trinitrotoluene altered the dielectric properties of the composite and in turn altered the SPR features. The change in the dielectric properties at the vicinity of the AuNPs affected the localized surface plasmon of the AuNPs, and consequently, was reflected by the plasmon coupling to the surface plasmon wave. That is, the coupling of the localized plasmon to the surface plasmon transduced the changes in the dielectric properties of the matrix as a result of the formation of the donor-acceptor complexes.

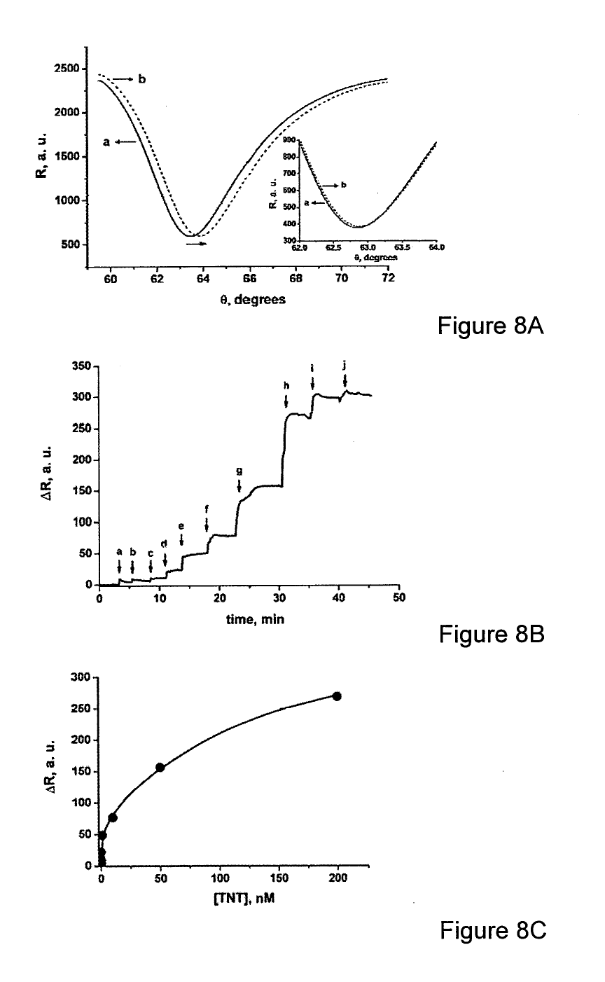

FIG. 8A shows the SPR curve of the bis-aniline-crosslinked AuNPs matrix before interaction with trinitrotoluene, curve (a), and after interaction with 200 nM of trinitrotoluene, curve (b). The minimum reflectivity angle of the spectrum is shifted by 0.4°. In a control experiment, a two-layer AuNPs matrix was assembled on the Au-coated glass surface by the primary association of thiopropionic acid-stabilized AuNPs (mean diameter 3.5 nm) on a cystamine-monolayer-functionalized gold slide, followed by the assembly of a second AuNPs layer by crosslinking the AuNPs to the base AuNPs layer with butane dithiol. Interaction of this AuNPs matrix with trinitrotoluene, 200 nM, did not yield any significant change in the SPR spectrum of the surface (FIG. 8A, inset), and only upon the interaction of the surface with 1 μM trinitrotoluene, a minute shift in the SPR spectrum was observed. Thus, when the bridging units lack electron donating properties, the SPR spectrum of the system is almost unaffected by the addition of trinitrotoluene. This is consistent with the primary assumption that the association of trinitrotoluene to the AuNPs surface occurs only upon the formation of π-donor-acceptor complexes between the bis-aniline units and trinitrotoluene (for further supporting evidence, vide infra).

FIG. 8(B) shows the sensogram corresponding to the reflectance changes upon the treatment of the bis-aniline crosslinked AuNPs matrix with variable concentrations of trinitrotoluene, and FIG. 8(C) depicts the corresponding calibration curve derived from the reflectance changes at θ=62.4°, upon interacting the bis-aniline-crosslinked AuNPs-modified electrode with variable concentrations of trinitrotoluene. The modified electrode enables the detection of the nitro compounds with a sensitivity corresponding to ca. 10 pM.

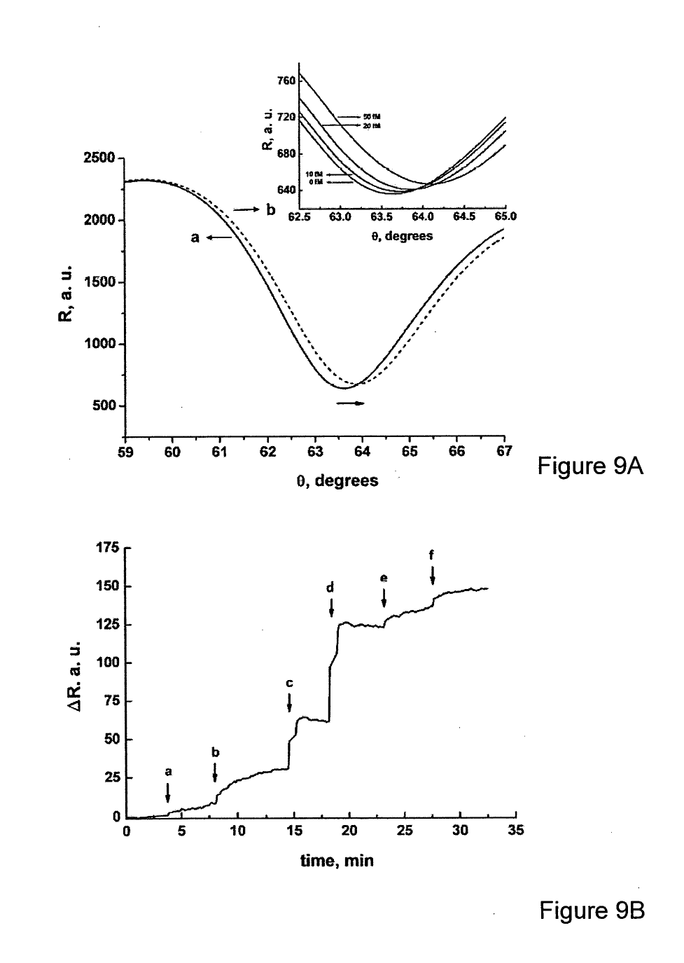

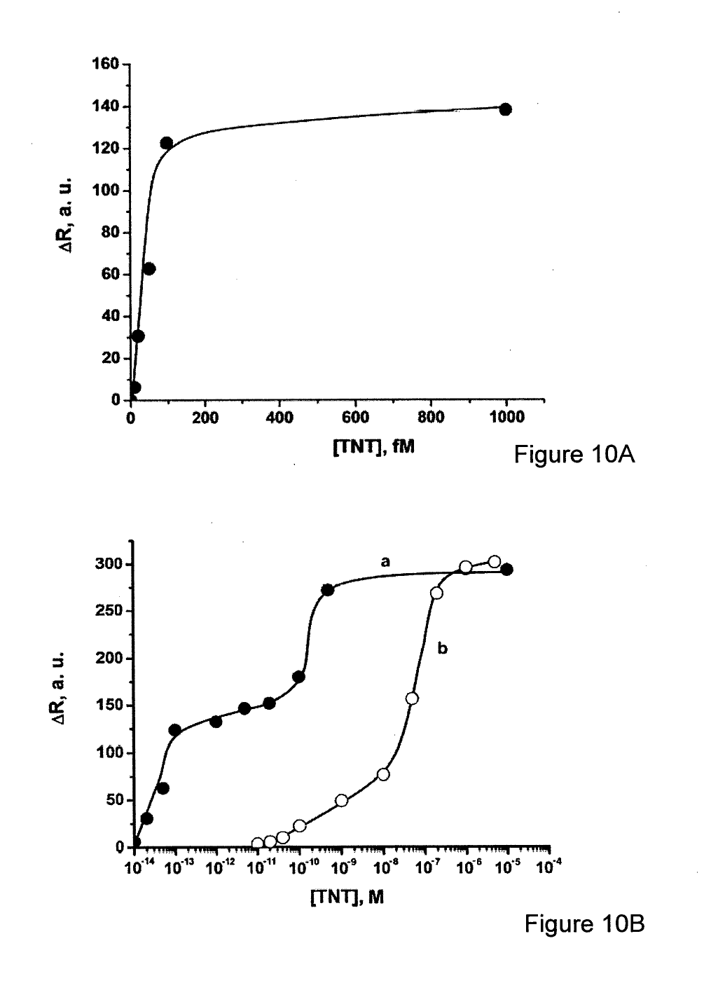

In the imprinting method disclosed above, the formation of the π-donor-acceptor complexes between the picric acid and the thioaniline-modified AuNPs, occurred during the electropolymerization on the Au-coated slide. The subsequent removal of the picric acid imprint molecules yielded molecular contours with optimal positioning of the π-donor sites for the association of the trinitrotoluene analyte. Indeed, as demonstrated the synergistic binding of trinitrotoluene to the AuNPs matrix by π-donor-acceptor interactions and molecular contours improved the association constant of trinitrotoluene to the matrix. FIG. 9(A) depicts the SPR curve of the imprinted crosslinked AuNPs composite before, curve (a), and after treatment with 1 pM of trinitrotoluene, curve (b). The SPR shift at this low concentration is comparable to the response of the non-imprinted matrix at 200 nM trinitrotoluene, FIG. 8A, indicating that an increased coverage of trinitrotoluene on the sensing surface occurred. FIG. 9(B) depicts the reflectance changes at the angle θ=62.7°, upon treating the imprinted sensing matrix with variable concentrations of trinitrotoluene. As the concentration of trinitrotoluene is elevated, the reflectance changes increase. FIG. 9C shows the derived calibration curve that corresponds to the analysis of trinitrotoluene by the imprinted matrix. The detection limit for the analysis of trinitrotoluene using the imprinted composite corresponds to a concentration of 10 fM trinitrotoluene that is ca. 10³-fold improved relative to the non-imprinted matrix. FIG. 10 depicts the comparison of the derived calibration curves for the imprinted, curve (a), and the non-imprinted, curve (b), matrices presented for clarity on a semi-logarithmic scale. The enhanced SPR shifts and the improved sensitivity for the detection of trinitrotoluene by the imprinted bis-anilinecrosslinked AuNPs matrix are attributed to the higher affinity of trinitrotoluene to the imprinted matrix, resulting in higher content of bound trinitrotoluene (vide infra).

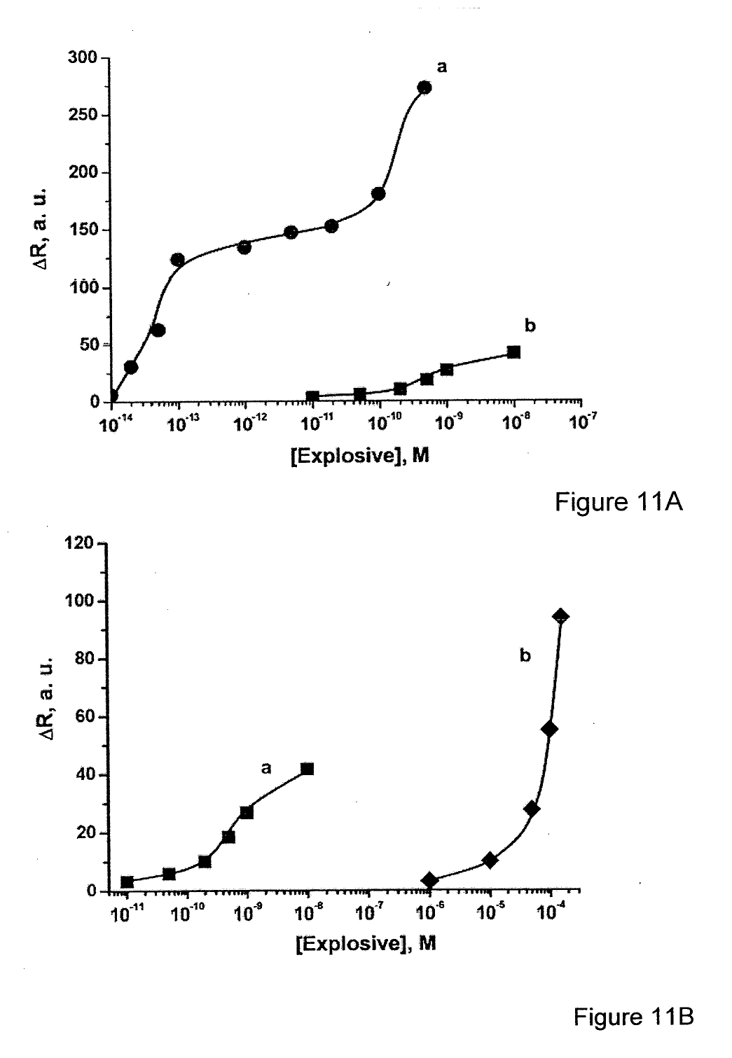

One may note that for the imprinted AuNPs composite two steps of changes in the reflectance values as a function of the concentration of added trinitrotoluene are observed. The first step is initiated at ca. 10 fM trinitrotoluene and the reflectance change (ΔR) saturates at ca. 5 pM. The second step of reflectance change is initiated at ca. 100 pM and the reflectance levels off at ca. 1 nM. In contrast, the non-imprinted matrix shows a single domain of ΔR that is initiated at ca. 10 pM and it reaches a saturation value at ca. 1 μM. The two-step reflectance calibration curve, FIG. 10, curve (a), is attributed to the existence of two types of trinitrotoluene binding sites. The reflectance changes at low trinitrotoluene concentrations (10 fM<[trinitrotoluene]<5 pM) are attributed to the association of trinitrotoluene to the imprinted donor sites of the matrix. The reflectance changes observed at higher trinitrotoluene concentrations (100 pM<[trinitrotoluene]<10 μM), that are also observed for the nonimprinted matrix, are attributed to the association of trinitrotoluene to the non-imprinted bis-aniline π-donor units. The specificity of the picric acid-imprinted polymer matrices toward the analysis of trinitrotoluene was further examined by subjecting imprinted AuNPs composite to 2,4-dinitrotoluene, DNT. FIG. 11(A) depicts the comparison between the calibration curves derived for trinitrotoluene, curve (a), and DNT, curve (b). While interacting the imprinted matrix with a bulk trinitrotoluene concentration of 10 fM yields detectable reflectance changes, comparable ΔR values upon interactions with DNT are observed only at bulk DNT concentrations above 50 pM. Also, the saturation values of ΔR for trinitrotoluene sensing (ΔR˜300 a.u.) are substantially higher than ΔR upon sensing of DNT (ΔR˜40 a.u.). These results are consistent with the fact that DNT exhibits substantially lower affinity towards the picric acid-imprinted matrix, as compared to trinitrotoluene. The lower affinity of DNT to the imprinted matrix defines the lower content of DNT associated with the matrix, resulting in a substantially lower detection limit and a lower response. While the imprinted AuNPs matrices sense trinitrotoluene in the femtomolar concentration range, these matrices respond to DNT only in the picomolar concentration range. That is, the analysis of trinitrotoluene by the imprinted AuNPs matrix is ca. 10³-fold more sensitive than the detection of DNT. This result is consistent with the fact that DNT exhibits decreased electron acceptor properties as compared to trinitrotoluene due to the lack of one of the electron attracting nitro groups. The lower electron affinity of DNT results in a weaker binding constant with the π-donor crosslinking units, and, thus, higher bulk concentrations are needed to yield a measurable SPR response. These conclusions are even further emphasized upon the analysis of 4-nitrotoluene, MNT, FIG. 11(B). The detection limit for analyzing mono-nitrotoluene, MNT is in the 10⁻⁵ M concentration range only, and no difference is observed upon sensing MNT by the imprinted or non-imprinted matrices. Evidently, the lack of two of the electron-withdrawing NO₂ groups weakens the electron acceptor features of MNT as compared to trinitrotoluene and DNT, and, thus, higher bulk concentrations of MNT are required to allow their association to the π-donor sites.

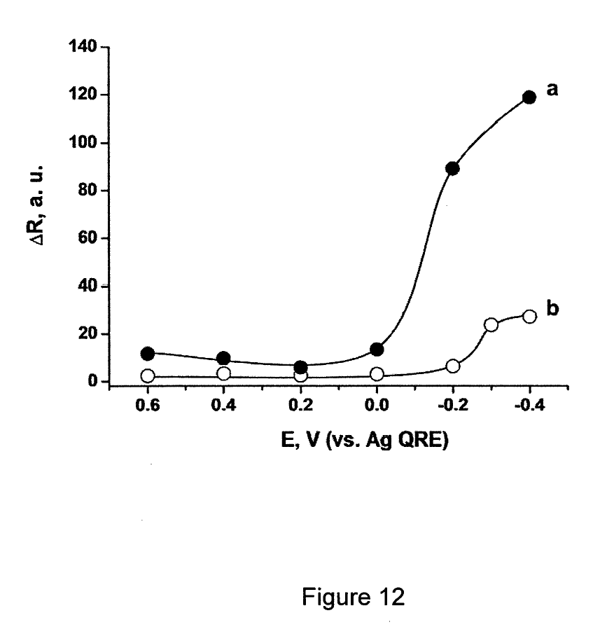

Further support that the successful ultra-sensitive detection of trinitrotoluene originates, indeed, from π-donor-acceptor interactions with the bis-aniline bridges was obtained by following the SPR reflectance changes of the imprinted, or non-imprinted, AuNPs crosslinked composites in the presence of trinitrotoluene (100 fM), upon applying an external potential to the Au substrate, FIG. 12, curves (a) and (b), respectively. A sharp change in the SPR reflectance intensities is observed at ca. −0.1 V (vs. a quasi-reversible Ag wire reference electrode). Prior to the addition of trinitrotoluene the potential scan on the electrode revealed only a minute reflectance change in the entire potential range, and specifically at −0.1 V vs. the Ag wire electrode, (<5 a.u. of reflectance). These results further support the formation of the π-donor-acceptor complexes in the bis-aniline-crosslinked AuNPs composite, and re-emphasize that the charge transfer process in the donor-acceptor complexes strongly affects the SPR spectra. In the potential region +0.6 to 0.0 V, the bridging units exist in their oxidized, quinoide, form. In this state, the bridges exhibit electron acceptor features, and thus the formation of the donor-acceptor complexes with trinitrotoluene is prohibited. At ca. −0.1 V, the bridging units are reduced to the bis-aniline π-donor form. In this configuration the formation of the π-donor-acceptor complex with trinitrotoluene proceeds, resulting in the changes in the SPR spectra. The increased changes in the reflectance intensities observed for the imprinted crosslinked AuNPs composite, (cf. FIG. 12, curve (a) as compared to (b)), are consistent with the enhanced binding of trinitrotoluene to the imprinted sites.

Further attempts were directed to characterize the structure and composition of the bis-anilinebridged AuNPs matrix. Ellipsometry was implemented as a spectroscopic tool. Ellipsometry is a powerful optical technique for the investigation of the dielectric properties of thin films and it has been widely used to characterize polymer layers and NP-polymer composites. In ellipsometric measurements the incident polarized monochromatic light beam is reflected from the sample. The reflected beam intensity and polarization changes are, then, measured, yielding the ellipsometric angles Ψ and Δ. These experimental quantities are compared with the values, calculated according to the suggested model describing the order of the layers on the surface, and a least-square value minimization is performed. The optical properties of 2-4 nm diameter AuNPs were recently studied through optical absorption and ellipsometric measurements and their dielectric function was found to nearly equal that of bulk gold in the spectral range of 207-414 nm. These observations allowed the integration of the bulk gold optical properties in the ellipsometric model for characterization of the electropolymerized AuNP matrices by fitting the ellipsometric data in the spectral range of 300-500 nm. By analyzing the ellipsometry results, the thickness of the thioaniline monolayer was estimated to be 1.1±0.2 nm, using a refractive index of n=1.56 for thioaniline. The thickness of the bis-aniline-crosslinked AuNP matrix was estimated by using the Effective Medium Approximation, based on the Maxwell-Garnett approach that describes the relationship between the effective dielectric function of the composite and the dielectric function and volume fraction of the metallic NPs. The estimated thickness of the crosslinked AuNPs composite was found to be 9.7±2.1 nm with a volume fraction of AuNPs corresponding to ca. 65±19%. Using the measured dimensions of the AuNPs (3.5 nm), this thickness translates to ca. three monolayers of AuNPs in the matrix. Further characterization of the composite was accomplished by electrochemical measurements. The AuNPs were synthesized with a molar ratio of thioaniline/ethane-sulfonic acid mixture (mercaptoethane sulfonate) that corresponded to 1:6. It is known that ca. 221 thiol molecules bind to a single AuNP with a core size of 4.0 nm. This value was re-calculated for the 3.5 nm AuNPs used in the present study, to yield a coverage of ca. 168 thiol molecules per single AuNP. Realizing that thioaniline provides only 1/7 of the total coverage, we estimate that a single AuNP is covered by ca. 24 thioanline units. The amount of the thioaniline units, electropolymerized in the AuNP composite, was further probed by the coulometric analysis of the cathodic redox wave at 0.05 V vs. SCE. The charge associated with the oxidation of the thioaniline units corresponded to 6×10⁻⁵ C·cm⁻², a value that translates to coverage of 3.8×10¹⁴ thioanilne molecules·cm⁻² in the composite. Realizing that each AuNP is covered by 24 thioaniline units, we estimate the surface coverage of the AuNPs to be 1.6×10¹³ particles cm⁻². Using the derived surface coverage of the AuNPs, and knowing the volume of a single AuNP, we estimate the thickness of the composite to be ca. 11.6 nm, assuming a random close packing model for the AuNPs in the matrix. This result suggests that the composite consists of ca. three random densely packed AuNPs layers, consistent with the ellipsometry results.

The association constant of trinitrotoluene binding to the bis-aniline-AuNPs units in the imprinted matrix and the content of imprinted sites in the matrix were calculated and to estimate the relative population of the sites bound by trinitrotoluene. The binding of trinitrotoluene to the π-donor binding sites in the imprinted and non-imprinted AuNPs matrices can be described by Eq. 4 and the Langmuir isotherm model. According to this model, the association constant of trinitrotoluene to the π-donor binding sites, is given by Eq. 3 where θ is the number of sites occupied by trinitrotoluene, and α is the total number of binding sites. At any given bulk concentration of trinitrotoluene, the value of θ can be evaluated by rearranging Eq. 3 in the form of Eq. 5.

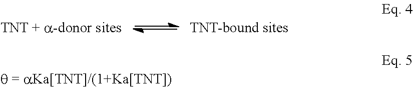

The double reciprocal of the Langmuir equation yields the Lineweaver-Burk relation, Eq. 3a.

From the values of slope and y-axis intercept the values of α and Kₐ can, then, be derived. Assuming that the reflectivity, R, is proportional to the number of trinitrotoluene-occupied sites, we analyzed the calibration curves for the non-imprinted and the imprinted matrices, FIGS. 8(C) and 9(C), respectively, according to the Lineweaver-Burk relation, Eq. 5. The derived association constants for the imprinted and the nonimprinted sites correspond to, Kₐ₁=6.4×10¹² M⁻¹, and K^{a2}=3.9×10⁹ M⁻¹, respectively. FIG. 10 presents the calibration curves derived for the imprinted and non-imprinted matrices on a semi-logarithmic scale. One can realize a two-step sensing response in the calibration curve for the imprinted matrix. We believe that this behavior is defined by two populations of binding sites which possess different association constants for trinitrotoluene binding, and correspond to the association to the imprinted and non-imprinted sites in the imprinted matrix. We use the Langmuir model for these two populations in order to describe the experimentally derived calibration curve. Assuming that the imprinted AuNPs matrix includes only two types of sites, imprinted and non-imprinted, then each of the independent sites binds trinitrotoluene according to Eq. 5, and the relation describing the coverage of trinitrotoluene in these two types of sites is given by Eq. 6.

θ=α₁K¹a[TNT]/(1+K¹a[TNT])+α₂K²a[TNT]/(1+K²a[TNT]) Eq. 6

The experimental calibration curve was, then, fitted to Eq. 5, allowing the extraction of Kₐ₁=1.7×10¹³ M⁻¹ and Kₐ₂=5.4×10⁹ M⁻¹, which correspond to the association constants of trinitrotoluene to the imprinted and non-imprinted sites, respectively. Also, the analysis indicated that the relative fraction of imprinted sites is ca. 48% of the total number of binding sites in the matrix. Evidently, both the Lineweaver-Burk analysis and the two populations fitting methods resulted in close values for each of the association constants between trinitrotoluene and the two types of binding sites associated with the sensing matrix. Thus, we will further use the mean values of: Kₐ₁=1.1×10¹³ M⁻¹, and Kₐ₂=4.7×10⁹ M⁻¹.

The lowest SPR detectable concentration of trinitrotoluene by the imprinted AuNPs matrix corresponded to 10 fM. Substitution of the derived values of association constants Kₐ₁ and Kₐ₂ into Eq. 5, yields the relative coverage of the trinitrotoluene occupied sites, θ/α, ca. 4.7%. Thus, at the lowest concentration detected, less than 5% of the total number of binding sites is occupied. In order to understand the effects of bound trinitrotoluene on the SPR shifts, and particularly, to realize the effect of the low detectable concentrations of trinitrotoluene on the SPR changes, we analyzed the experimental curves. The SPR curve is characterized by three major values: θₚ, Γ_{w} and Rₘᵢₙ, where θₚ and Rₘᵢₙ correspond to the minimum reflectivity plasmon angle and to the reflectance at this angle, respectively, and Γ_{w} is the width of the SPR curve at half of the maximum reflectance intensity. The experimental results reveal that the binding of trinitrotoluene results in changes in Rₘᵢₙ, θₚ and Γ_{w}. Accordingly, we fitted the different SPR spectra in a reduced range of SPR angles (±1.5° around the plasmon angle), by using Frenel's equations based five-layers model and using a Winspall 2.0 program. The derived values for the real and imaginary parts of the complex index of refraction, n=n_{R}+ik, for the different curves were, then, translated into the real and imaginary parts of the complex dielectric constant values, ∈′ and ∈″, respectively. Eq. 7 relates the complex index of refraction to the complex dielectric function, ∈=∈′+∈″.

∈=n²;(5a) ∈′=n_{R}²−k²;(5b) ∈″=2n_{R}k, Eq. 7

The results for the fitting of the SPR curves corresponding to the Au layer-coated SPR slide, the thioaniline monolayer-modified surface, the surface after the stepwise deposition of the bis-anilinebridged AuNPs composite, and the modified surface treated with variable concentrations of trinitrotoluene are summarized in Table 2.

TABLE 2
Parameters derived by fitting the experimental SPR curves.

| | d, nm | n | k | ε′ | ε″ | σ, S cm⁻¹ |
|---|---|---|---|---|---|---|
| Bare Au/Cr | 54.3 | 0.186 | 3.91 | −13.92 | 1.39 | |
| Thioaniline SAM | 1.29 | 1.68 | 0 | 2.82 | | |
| AuNPs matrix | 7.03 | 0.1546 | 2.61 | −6.79 | 0.81 | 207 |
| TNT 5 fM | | 0.1601 | 2.58 | −6.64 | 0.824 | 212 |
| TNT 10 fM | | 0.1616 | 2.57 | −6.60 | 0.83 | 213 |
| TNT 20 fM | | 0.1665 | 2.52 | −6.30 | 0.84 | 215 |
| TNT 50 fM | | 0.1834 | 2.43 | −5.87 | 0.89 | 228 |

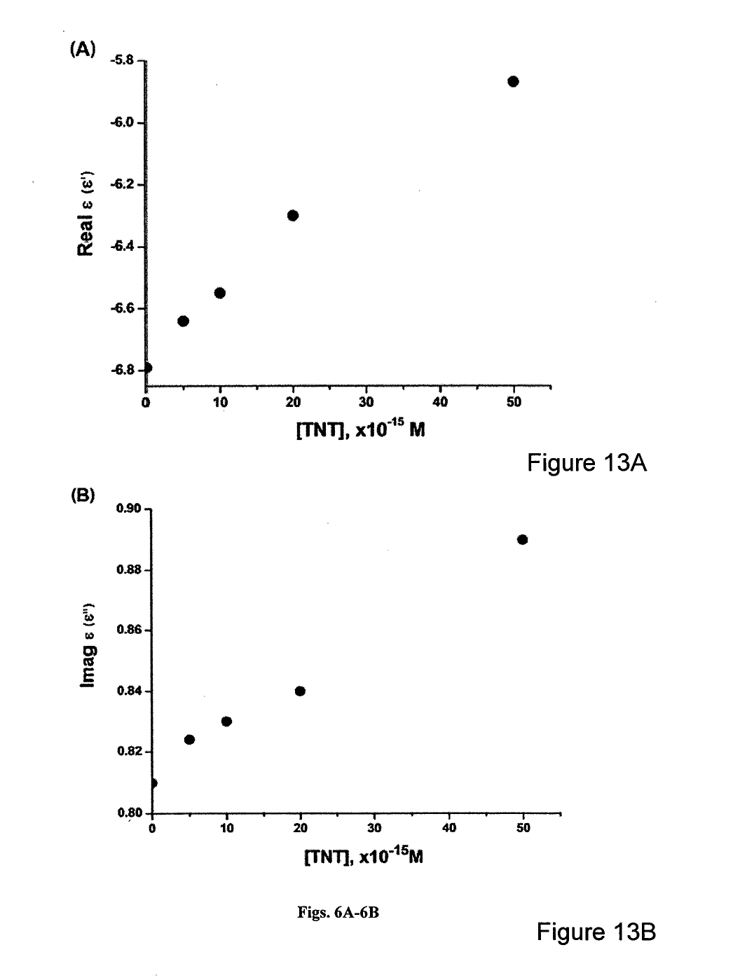

The values of the dielectric constants for the Au-coated SPR slide are very close to the reported values for bulk gold ∈′=−13.4; ∈″=1.4, and provide a further support for the fitting procedure. FIGS. 13 (A) and (B) depict the calculated values of the real part, ∈′, and of the imaginary part, ∈″, of the dielectric constant associated with the bis-aniline-crosslinked AuNPs matrix in the presence of variable concentrations of trinitrotoluene. The values of the dielectric constant components are smaller than those of bulk Au and are strongly affected by the association of trinitrotoluene, with the formation of the respective donor-acceptor complexes. The real part of the dielectric constant, ∈′, for the AuNPs exhibits values that are two-fold lower than the corresponding value for bulk gold. Upon the increase of the trinitrotoluene concentration and the formation of the respective π-donor-acceptor complexes the real part of the dielectric constant increases and becomes less negative. The same tendency is observed for the imaginary part of the dielectric constant, ∈″, that increases as the coverage of trinitrotoluene is elevated. For example, at a bulk trinitrotoluene concentration of 50 fM, ∈″ increases by 10%. The changes in ∈″ are possibly connected to the electrical conductivity of the bis-aniline-AuNPs matrix. According to the semiclassical Drude model for metal-like conductor, the electrical conductivity in the optical frequencies range is coupled to its optical properties. The conducting material is characterized by the complex dielectric function, ∈, and in the presence of an incident light of wavelength λ, the conductivity, σ, is given by Eq. 8, where ∈″ is the imaginary part of the dielectric constant, c is the speed of light, and ∈₀ is the free-space permittivity.

∈″=i2σλ/c∈₀ Eq. 8

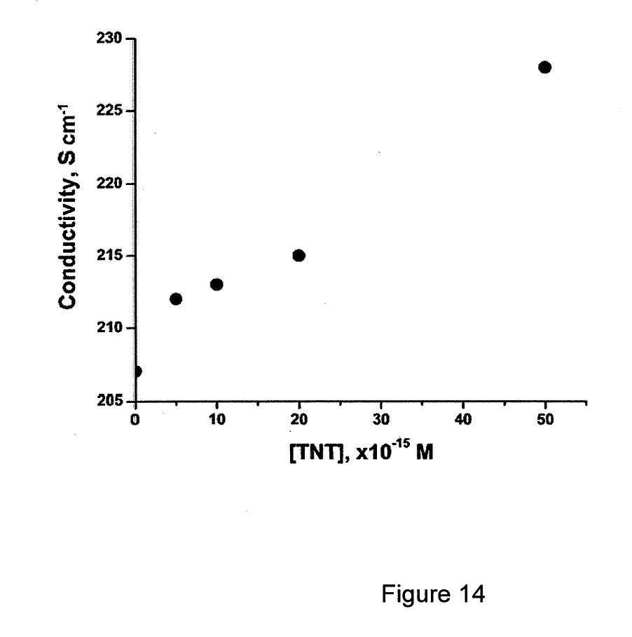

It may be seen from this equation, that a variation of the conductivity of the layer can cause a change in the imaginary part of the dielectric constant. The fitting of the SPR curves, upon the association of trinitrotoluene, revealed that the imaginary part of the dielectric constant increases as the trinitrotoluene concentration is elevated. This implies that the conductivity of the AuNPs matrix increases, too, as the coverage of trinitrotoluene becomes higher. FIG. 14 presents the calculated values of the conductivity changes as a function of the trinitrotoluene concentration. Our analysis shows that the electrical conductivity of the imprinted bis-aniline-crosslinked AuNPs matrix in the absence of trinitrotoluene is ca. 200 S cm⁻¹, and upon the association of 50 fM trinitrotoluene with the matrix, the conductivity changes by ca. 10%, FIG. 14. This conductivity change implies on the origin of the successful ultrasensitive detection of trinitrotoluene by the AuNPs composite. That is, at a trinitrotoluene concentration of 50 fM, and according to Eq. 5 and the derived values of the association constants, we estimate that ca. 17% of the binding sites are occupied by trinitrotoluene molecules. Albeit very few recognition events occur in the matrix, the electronic perturbations at the inter-particle bis-aniline bridges translate into a collective conductivity change of the whole composite. Presumably, the localized charge transfer between the π-donor and acceptor units alters the dielectric function and therefore the conductivity of the interconnected nanoparticle composite resulting in the SPR curve changes.

It should be noted that recent measurements for the truly metallic state of polyaniline reported conductivities in the order of 103 S cm⁻¹, in accordance with the predictions of the Drude theory. Also, the electrical conductivity of thin π-conjugated polymer layers was recently evaluated by SPR and a value of 600 S cm⁻¹ was estimated, which is in a good agreement with the experimental value of 500 S cm⁻¹, measured by the widely used Van der Pauw method. These results support our conclusion that the ultrasensitive, label-free, SPR method for analyzing trinitrotoluene originates, indeed, from conductivity changes within the bis-aniline-crosslinked AuNP matrix, as a result of the association of trinitrotoluene. It should be noted regarding the formation of a donor-acceptor complex between tetracyanoethylene (acceptor) and a monolayer of a tetramethyl xylyl dithiol (donor), that a 50-fold increase in the conductivity through the monolayer was observed upon the formation of the complex, consistent with our analysis.

In order to highlight the importance of the present label-free SPR method for the detection of trinitrotoluene, the sensitivity of this method (detection limit ca. 1.2×10⁻³ ppt) was compared to other reported trinitrotoluene detection methods, Table 3. As may be noted, the present method is at least 10³-fold more sensitive than any previously reported method.

TABLE 3
Comparison of different trinitrotoluene sensors.

| Detection Method | detection limit |
|---|---|
| Remote microelectrode electrochemical sensor in water | 50 ppb |
| Luminiscent oligo(tetraphenyl)silole nanoparticles | 20 ppb |
| Electrochemical detection by carbon nanotubes | 5 ppb |
| Biochip (on Au) via QCM or SPR | 1 ppb-10 ppb |
| Electrochemical detection using metallic NP-CNT | 1 ppb |
| composites | |
| Adsorptive stripping by carbon nanotubes-modified GCE | 600 ppt |
| Electrochemical detection by mesoporous SiO₂-modified | 414 ppt |
| electrodes | |
| Oligo(ethylene glycol)-based SPR | 80 ppt |
| Electrochemical sensing by imprinted electropolymerized | 46 ppt |
| bis-aniline-crosslinked AuNPs | |
| SPR, fabricated dinitrophenylated-keyhole lympet | 5 ppt |
| hemocyanin (DNP-KLH) protein conjugate | |
| Indirect competitive immunoassay using SPR | 2 ppt |
| SPR sensing by bis-aniline-crosslinked picric | 1.2 × 10⁻³ |
| acid-imprinted Au-Nanoparticles composite | (present study) |

1.-50. (canceled)

51. A method for determining the presence and/or concentration of analyte molecules in a sample, said method comprising:
contacting a matrix of a plurality of transition metal nanoparticles (TMNPs), each carrying a plurality of recognition groups, with a sample suspected of containing analyte molecules; said recognition groups being capable of undergoing a physical and/or chemical interaction with said analyte molecules; wherein said TMNPs are associated with the recognition groups via at least one reactive group selected from —S, —NH₂ and —CO₂⁻; and wherein said matrix comprises analyte-recognition fields complementary to the shape or size of said analyte molecule and
monitoring at least one of a chemical and a physical change in said matrix resulting from an interaction between said analyte molecules and said recognition groups;
wherein said at least one of a chemical and a physical change is indicative of at least one of presence and quantity of said analyte in the sample.

52. The method according to claim 51, wherein said analyte is selected from trinitrotoluene (TNT), a nitro compound or a combination thereof.

53. The method according to claim 51, wherein each TMNP in said plurality of TMNPs is associated with each other through a plurality of recognition groups.

54. The method according to claim 51, wherein said recognition groups are selected to be capable of undergoing chemical and/or physical interaction with said analyte molecules, said interaction is reversible or permanent.

55. The method according to claim 56, wherein said interaction is via one or more of a single bond, a double bond, a triple bond, van der Waals bonding, hydrogen bonding, π-stacking interaction, electrostatic interaction, complexation and caging.

56. The method according to claim 51, wherein said TMNPs are nanoparticles of at least one transition metal selected from the d-block of the Periodic Table of the Elements.

57. The method according to claim 56, wherein said nanoparticles are of a metal selected from platinum (Pt), palladium (Pd), iridium (Ir), gold (Au), silver (Ag), nickel (Ni) and titanium (Ti), or any alloy of any of said metals.

58. The method according to claim 57, wherein said TMNPs are gold nanoparticles or contain gold metal.

59. The method according to claim 51, wherein —S is a sulfur containing group.

60. The method according to claim 59, wherein said sulfur containing group is selected from thioaniline, thioaniline dimer and oligomers thereof, each of said groups having one or more sulfur groups.

61. The method according to claim 60, wherein said one or more sulfur groups are selected from p-thioaniline and the oligothianilines having 2, 3, 4, 5, 6, 7, 8, 9 or 10 p-thioaniline monomer units.

62. The method according to claim 61, wherein the recognition groups are thioaniline dimer 4-amino-3-(4-mercaptophenylamino)benzenthiol.

63. The method according to claim 51, wherein said active surface is conductive, preferably selected from an electrode and a metal (or alloy) coated glass.

64. The method according to claim 51, wherein the matrix is associated with an active surface through one or more binding moieties, said binding moieties being the same or different from the recognition groups used to associate the plurality of TMNP in the matrix.

65. The method according to claim 64, wherein said binding moieties are thioaniline.

66. The method according to claim 51, wherein the interaction between the matrix and the analyte molecules is probed by monitoring at least one measurable change in at least one property or structure of the target molecule or one or more component of the matrix, wherein said measurable change is in any one electric property, electrochemical property or spectroscopic property.

67. The method according to claim 65, wherein said measurable change is monitored by voltammetric or SPR measurements.

68. The method according to claim 51, comprising
(a) providing nanoparticles of a transition metal, said nanoparticles carrying a plurality of recognition groups capable of undergoing interaction with analyte molecules;
(b) contacting said nanoparticles with a sample suspected of containing analyte molecules;
(c) providing assay conditions to permit interaction between said recognition groups and the analyte molecule(s); and
(d) probing the interaction to thereby detect at least one change in at least one dielectric property in the vicinity of the nanoparticles, whereby said change is indicative of at least the presence and quantity of said analyte molecule(s) in the sample.

69. An electrode comprising a conductive surface and being connected to a matrix, said matrix comprising a plurality of transition metal nanoparticles (TMNPs), wherein substantially each of said nanoparticles is connected to another by at least one recognition group capable of mediating electron transfer between nanoparticles of the matrix; at least a portion of said plurality of nanoparticles is connected to said conductive surface by at least one surface binding group, capable of mediating electron transfer between the matrix and said conductive surface.

70. A device for carrying out a detection of an analyte in a sample, said device comprising at least one assay unit having a matrix of a plurality of transition metal nanoparticles (TMNPs), each carrying a plurality of recognition groups, said recognition groups being capable of undergoing a physical and/or chemical interaction with said analyte molecules; wherein said TMNPs are associated with the recognition groups via at least one reactive group selected from —S, —NH₂ and —CO₂⁻; and wherein said matrix comprises analyte-recognition fields complementary to the shape or size of said analyte molecule; and means for probing at least one of a chemical and a physical change in said matrix resulting from an interaction between said analyte molecules and said recognition groups.
